(19) United States
(12) Patent Application Publication
(10) Pub. No.: US 2003/0186280 A1
(43) Pub. Date: Oct. 2, 2003

(54) Methods for detecting genomic regions of biological significance

(75) Inventor: Giulia C. Kennedy, San Francisco, CA (US)

Correspondence Address: AFFYMETRIX, INC, ATTN: CHIEF IP COUNSEL, LEGAL DEPT., 3380 CENTRAL EXPRESSWAY, SANTA CLARA, CA, 95051, US

(73) Assignee: Affymetrix, INC., Santa Clara, CA

(21) Appl. No.: 10/316,629

(22) Filed: Dec. 10, 2002

Related U.S. Application Data
(60) Provisional application No. 60/369,019, filed on Mar. 28, 2002.
(60) Provisional application No. 60/392,406, filed on Jun. 26, 2002.
(60) Provisional application No. 60/412,491, filed on Sep. 20, 2002.
(60) Provisional application No. 60/392,305, filed on Jun. 26, 2002.
(60) Provisional application No. 60/393,668, filed on Jul. 3, 2002.

(51) Int. Cl. C12Q 1/68; G06F 19/00; G01N 33/48; G01N 33/50

(52) U.S. Cl. 435/6; 702/20

(57) ABSTRACT

In one embodiment of the invention, methods are provided for identifying a genomic region under natural selection. The methods include genotyping at least 5,000 SNPs in at least two populations; determining difference of allele frequencies between the populations to identify at least one SNP with a Fst value of at least 0.3; identifying the genomic region where the at least one SNP resides as a putative genomic region under natural selection.

### RELATED APPLICATIONS

This application claims the priority of U.S. Provisional Application Nos. 60/369,019, filed on Mar. 28, 2002, No. 60/392,406, filed on Jun. 26, 2002, No. 60/412,491, filed on Sep. 20, 2002, No. 60/392, 305, filed on Jun. 26, 2002, No. 60/393,668, filed on Jul. 3, 2002. All cited provisional applications are incorporated herein by reference.

This application is also related to U.S. patent application No. 09/916,135, filed on Jul. 25, 2001, which is incorporated herein by reference for all purposes. Introduction

Natural selection is at the heart of adaptive evolution. Characteristics of human populations are shaped by their responses to pathogens, diet, climate and other selective pressures. Identifying regions of the genome under directional selection has important implications for understanding our evolution as a species.

### SUMMARY OF THE INVENTION

In one aspect of the invention, methods are provided to detect genes or genomic regions of biological importance. In some embodiments, genomic samples from different (at least two) populations (a population, as used herein, refers to a race, people from a geographic region, etc.) are examined for differences in allele frequencies. In preferred embodiments, differences in SNP allele frequencies are examined. A whole genome assay (WGA) developed at Affymetrix, Inc. (Santa Clara, Calif.), described in, e.g., U.S. Provisional Application, attorney docket number 3504, and U.S. Pat. No. 09/916,135, provides an efficient method for genotyping a large number of SNPs in a complex DNA sample. Other methods for genotyping may also be suitable for the methods of the invention.

The differences in frequencies may be evaluated using the Fst statistic (see below). The degree of geographic structure can be estimated by the F_{ST} statistic (Weir, B. S. Genetic Data Analysis II, Sinauer Associates, Inc Sunderland, Mass. (1996)), which varies from 0 to 1.

Regions of the genome that are under selective pressure may be identified according to the allele difference, e.g., calculating F_{ST} values for all SNPs and looking for SNPs with exceptionally high values, such as greater than 0.3, 0.4, 0.5, 0.6, 0.7, or 0.8.

The genomic region under selective pressure may be identified as having potential biological functions, such as disease resistance.

In some embodiments, genes residing in the genomic regions may be identified by, for example, examining gene annotation databases.

The genomic region or the genes residing in the genomic regions may be further examined and used as therapeutical targets, as they have already been shown by human history to be targets of regulation.

One of skill in the art would appreciate that the methods of the invention is not limited to any particularly species. The methods can be used detect genomic regions of interest in many organisms such as human, other animals, plants, etc.

### BRIEF DESCRIPTION OF THE FIGURES

The accompanying drawings, which are incorporated in and form a part of this specification, illustrate embodiments of the invention and, together with the description, serve to explain the embodiments of the invention:

FIG. 1 Fragment Selection by PCR (FSP). Digestion of genomic DNA with a restriction enzyme (e.g. Bg1II), results in fragments of various sizes (black), including fragments 400-800 bp long (red). Adaptors are ligated to all size fragments, but only those fragments in the 400-800 bp size range are amplified. The amplified target is fragmented and labeled and hybridized to synthetic DNA microarrays.

FIG. 2 Hybridized chip images a.. Microarray hybridized to reduced complexity (˜4×10⁷ bp) biotin-labeled DNA b. Microarray hybridized with biotin-labeled human genomic DNA (3×10⁹ bp). Signals from hybridization controls are detected. c. SNP miniblock showing hybridization of FSP target in three individuals, demonstrating the three possible genotypes; AA (left), AB (middle) and BB (right). Probes are synthesized as perfect match (PM) 25-mers, and as one-base mismatches (MM) in the center. Probes for both A and B alleles, on both sense and antisense strands are synthesized, for a total of 56 probes per SNP miniblock.

FIG. 3 Cluster visualization of SNPs. Relative allele signal (RAS) is calculated for each sample on both strands and plotted in two dimensions, demonstrating various types of clustering properties: a. SNP with ideal clustering properties; b. SNP forming 3 distinct clusters in the sense, but not antisense, dimension; c. Poorly clustering SNP; d. This SNP forms two well-separated and tight clusters; genotyping of additional samples may reveal instances of the minor allele homozygote (in this case, BB).

FIG. 4 Inter-SNP distances on Golden Path. The SNP map positions were determined by TSC on the April 2002 release of the Golden Path (NCBI Build 29). The distances between markers, in kb, are plotted as a frequency distribution. The cumulative % of markers is indicated by the dotted line

FIG. 5a Distribution of heterozygosity in three populations. The frequency of heterozygotes for each SNP was determined in 3 populations and plotted as a distribution across 10 bins, plus an additional category for SNPs that showed zero heterozygotes in that population, ie monomorphic SNPs (leftmost bars).

FIG. 5b Distribution of pairwise F_{ST} values in three populations. Pairwise allele frequency comparisons in three human populations. A total of 13,647 SNPs were scored in 20 individuals per group. F_{ST} values were calculated as previously described¹⁶. Resulting values were ranked from lowest to highest F_{ST} value for each pairwise comparison and denoted K(i). K(i) corresponds to the SNP that yields the i^{th} largest F_{ST} value and is population specific.

FIG. 5c Overlap in high F_{ST} SNPs among three populations. Pairwise F_{ST} values were calculated as in FIG. 5b. Venn diagram shows numbers of SNPs with F_{ST} values ≧0.40 in each overlap.

FIG. 5d Tajima D values for SNPs in three populations. Tajima D was calculated on 13,647 SNPs for all three populations. An average shifted histogram (ASH) technique was used to generate smooth density estimations for each population⁴¹. The built-in R (version 1.5.1) function “density” was applied. The default normal kernel is used with an automatic chosen bandwidth for each population⁴².

FIG. 6 Percentage ancestral allele as a function of allele frequency in three populations. Genotypes were determined for chimp and gorilla and the percent A allele was calculated for each frequency bin. As in FIG. 7, the “A” allele for each SNP was determined alphabetically

FIG. 7 Percentage non-ancestral allele as a function of F_{ST}. For each of the three populations, SNPs with allele A frequencies >0.8 were selected, F_{ST} values for each of the pairwise comparisons was calculated, binned and the percentage of non-ancestral (ie B) alleles determined for each bin. FIGS. 8a-d Mean Tajima D as a function of F_{ST} across three populations. Two-way (a-c) and three-way (d) F_{ST} values were calculated, as described ¹⁶. Tajima's D statistic was calculated for all SNPs³⁵. The plots were generated by kernel smoothing, a method that can be viewed as a locally weighted average for a continuously shifting window. The locally assigned weights sum to unity and die off smoothly when they move away from the center target point⁴². This method provides a smooth curve and summarizes the trend of Tajima D as a function of F_{ST} values. The plots show an overall negative association between Tajima D and F_{ST} values. This association is weak when F_{ST} is small; it becomes significant when F_{ST} is between 0.3 and 0.7. The association is unstable when F_{ST} is >0.7, mainly due to the small number of sample points in that region.

### DETAILED DESCRIPTION OF THE EMBODIMENTS OF THE INVENTION

The present invention has many preferred embodiments and relies on many patents, applications and other references for details known to those of the art. Therefore, when a patent, application, or other reference is cited or repeated below, it should be understood that it is incorporated by reference in its entirety for all purposes as well as for the proposition that is recited.

I. General

As used in this application, the singular form “a,” “an,” and “the” include plural references unless the context clearly dictates otherwise. For example, the term “an agent” includes a plurality of agents, including mixtures thereof.

An individual is not limited to a human being but may also be other organisms including but not limited to mammals, plants, bacteria, or cells derived from any of the above.

Throughout this disclosure, various aspects of this invention can be presented in a range format. It should be understood that the description in range format is merely for convenience and brevity and should not be construed as an inflexible limitation on the scope of the invention. Accordingly, the description of a range should be considered to have specifically disclosed all the possible subranges as well as individual numerical values within that range. For example, description of a range such as from 1 to 6 should be considered to have specifically disclosed subranges such as from 1 to 3, from 1 to 4, from 1 to 5, from 2 to 4, from 2 to 6, from 3 to 6 etc., as well as individual numbers within that range, for example, 1, 2, 3, 4, 5, and 6. This applies regardless of the breadth of the range.

The practice of the present invention may employ, unless otherwise indicated, conventional techniques and descriptions of organic chemistry, polymer technology, molecular biology (including recombinant techniques), cell biology, biochemistry, and immunology, which are within the skill of the art. Such conventional techniques include polymer array synthesis, hybridization, ligation, and detection of hybridization using a label. Specific illustrations of suitable techniques can be had by reference to the example herein below. However, other equivalent conventional procedures can, of course, also be used. Such conventional techniques and descriptions can be found in standard laboratory manuals such as Genome Analysis: A Laboratory Manual Series (Vols. I-IV), Using Antibodies: A Laboratory Manual, Cells: A Laboratory Manual, PCR Primer: A Laboratory Manual, and Molecular Cloning: A Laboratory Manual (all from Cold Spring Harbor Laboratory Press), Stryer, L. (1995) Biochemistry (4th Ed.) Freeman, New York, Gait, “Oligonucleotide Synthesis: A Practical Approach” 1984, IRL Press, London, Nelson and Cox (2000), Lehninger, Principles of Biochemistry 3rd Ed., W. H. Freeman Pub., New York, N.Y. and Berg et al. (2002) Biochemistry, 5th Ed., W. H. Freeman Pub., New York, N.Y., all of which are herein incorporated in their entirety by reference for all purposes.

The present invention can employ solid substrates, including arrays in some preferred embodiments. Methods and techniques applicable to polymer (including protein) array synthesis have been described in U.S. Ser. No. 09/536,841, WO 00/58516, U.S. Pat. Nos. 5,143,854, 5,242,974, 5,252,743, 5,324,633, 5,384,261, 5,405,783, 5,424,186, 5,451,683, 5,482,867, 5,491,074, 5,527,681, 5,550,215, 5,571,639, 5,578,832, 5,593,839, 5,599,695, 5,624,711, 5,631,734, 5,795,716, 5,831,070, 5,837,832, 5,856,101, 5,858,659, 5,936,324, 5,968,740, 5,974,164, 5,981,185, 5,981,956, 6,025,601, 6,033,860, 6,040,193, 6,090,555, 6,136,269, 6,269,846 and 6,428,752, in PCT Applications Nos. PCT/US99/00730 (International Publication Number WO 99/36760) and PCT/US01/04285, which are all incorporated herein by reference in their entirety for all purposes.

Patents that describe synthesis techniques in specific embodiments include U.S. Pat. Nos. 5,412,087, 6,147,205, 6,262,216, 6,310,189, 5,889,165, and 5,959,098. Nucleic acid arrays are described in many of the above patents, but the same techniques are applied to polypeptide arrays.

Nucleic acid arrays that are useful in the present invention include those that are commercially available from Affymetrix (Santa Clara, Calif.) under the brand name GeneChip®. Example arrays are shown on the website at affymetrix.com. The present invention also contemplates many uses for polymers attached to solid substrates. These uses include gene expression monitoring, profiling, library screening, genotyping and diagnostics. Gene expression monitoring, and profiling methods can be shown in U.S. Pat. Nos. 5,800,992, 6,013,449, 6,020,135, 6,033,860, 6,040,138, 6,177,248 and 6,309,822. Genotyping and uses therefore are shown in U.S. Ser. Nos. 60/319,253, 10/013,598, and U.S. Pat. Nos. 5,856,092, 6,300,063, 5,858,659, 6,284,460, 6,361,947, 6,368,799 and 6,333,179. Other uses are embodied in U.S. Pat. Nos. 5,871,928, 5,902,723, 6,045,996, 5,541,061, and 6,197,506.

The present invention also contemplates sample preparation methods in certain preferred embodiments. Prior to or concurrent with genotyping, the genomic sample may be amplified by a variety of mechanisms, some of which may employ PCR. See, e.g., PCR Technology: Principles and Applications for DNA Amplification (Ed. H. A. Erlich, Freeman Press, NY, N.Y., 1992); PCR Protocols: A Guide to Methods and Applications (Eds. Innis, et al., Academic Press, San Diego, Calif., 1990); Mattila et al., Nucleic Acids Res. 19, 4967 (1991); Eckert et al., PCR Methods and Applications 1, 17 (1991); PCR (Eds. McPherson et al., IRL Press, Oxford); and U.S. Pat. Nos. 4,683,202, 4,683,195, 4,800,159 4,965,188,and 5,333,675, and each of which is incorporated herein by reference in their entireties for all purposes. The sample may be amplified on the array. See, for example, U.S. Pat. No. 6,300,070 and U.S. patent application No. 09/513,300, which are incorporated herein by reference.

Other suitable amplification methods include the ligase chain reaction (LCR) (e.g., Wu and Wallace, Genomics 4, 560 (1989), Landegren et al., Science 241, 1077 (1988) and Barringer et al. Gene 89:117 (1990)), transcription amplification (Kwoh et al., Proc. Natl. Acad. Sci. USA 86, 1173 (1989) and WO88/10315), self sustained sequence replication (Guatelli et al., Proc. Nat. Acad. Sci. USA, 87, 1874 (1990) and WO90/06995), selective amplification of target polynucleotide sequences (U.S. Pat. No. 6,410,276), consensus sequence primed polymerase chain reaction (CP-PCR) (U.S. Pat. No. 4,437,975), arbitrarily primed polymerase chain reaction (AP-PCR) (U.S. Pat. Nos. 5,413,909, 5,861,245) and nucleic acid based sequence amplification (NABSA). (See, U.S. Pat. Nos. 5,409,818, 5,554,517, and 6,063,603, each of which is incorporated herein by reference). Other amplification methods that may be used are described in, U.S. Pat. Nos. 5,242,794, 5,494,810, 4,988,617 and in U.S. Ser. No. 09/854,317, each of which is incorporated herein by reference.

Additional methods of sample preparation and techniques for reducing the complexity of a nucleic sample are described in Dong et al., Genome Research 11, 1418 (2001), in U.S. Pat. Nos. 6,361,947, 6,391,592 and U.S. Patent application Nos. 09/916,135, 09/920,491, 09/910,292, and 10/013,598.

Methods for conducting polynucleotide hybridization assays have been well developed in the art. Hybridization assay procedures and conditions will vary depending on the application and are selected in accordance with the general binding methods known including those referred to in: Maniatis et al. Molecular Cloning: A Laboratory Manual (2nd Ed. Cold Spring Harbor, N.Y., 1989); Berger and Kimmel Methods in Enzymology, Vol. 152, Guide to Molecular Cloning Techniques (Academic Press, Inc., San Diego, Calif., 1987); Young and Davism, P.N.A.S, 80: 1194 (1983). Methods and apparatus for carrying out repeated and controlled hybridization reactions have been described in U.S. Pat. Nos. 5,871,928, 5,874,219, 6,045,996 and 6,386,749, 6,391,623 each of which are incorporated herein by reference.

The present invention also contemplates signal detection of hybridization between ligands in certain preferred embodiments. See U.S. Pat. Nos. 5,143,854, 5,578,832; 5,631,734; 5,834,758; 5,936,324; 5,981,956; 6,025,601; 6,141,096; 6,185,030; 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

Methods and apparatus for signal detection and processing of intensity data are disclosed in, for example, U.S. Pat. Nos. 5,143,854, 5,547,839, 5,578,832, 5,631,734, 5,800,992, 5,834,758; 5,856,092, 5,902,723, 5,936,324, 5,981,956, 6,025,601, 6,090,555, 6,141,096, 6,185,030, 6,201,639; 6,218,803; and 6,225,625, in U.S. Patent application 60/364,731 and in PCT Application PCT/US99/06097 (published as WO99/47964), each of which also is hereby incorporated by reference in its entirety for all purposes.

The practice of the present invention may also employ conventional biology methods, software and systems. Computer software products of the invention typically include computer readable medium having computer-executable instructions for performing the logic steps of the method of the invention. Suitable computer readable medium include floppy disk, CD-ROM/DVD/DVD-ROM, hard-disk drive, flash memory, ROM/RAM, magnetic tapes and etc. The computer executable instructions may be written in a suitable computer language or combination of several languages. Basic computational biology methods are described in, e.g. Setubal and Meidanis et al., Introduction to Computational Biology Methods (PWS Publishing Company, Boston, 1997); Salzberg, Searles, Kasif, (Ed.), Computational Methods in Molecular Biology, (Elsevier, Amsterdam, 1998); Rashidi and Buehler, Bioinformatics Basics: Application in Biological Science and Medicine (CRC Press, London, 2000) and Ouelette and Bzevanis Bioinformatics: A Practical Guide for Analysis of Gene and Proteins (Wiley & Sons, Inc., 2nd ed., 2001).

The present invention may also make use of various computer program products and software for a variety of purposes, such as probe design, management of data, analysis, and instrument operation. See, U.S. Pat. Nos. 5,593,839, 5,795,716, 5,733,729, 5,974,164, 6,066,454, 6,090,555, 6,185,561, 6,188,783, 6,223,127, 6,229,911 and 6,308,170.

Additionally, the present invention may have preferred embodiments that include methods for providing genetic information over networks such as the Internet as shown in U.S. Patent applications 10/063,559, 60/349,546, 60/376,003, 60/394,574, 60/403,381.

II. Glossary

The following terms are intended to have the following general meanings as there used herein.

Nucleic acids according to the present invention may include any polymer or oligomer of pyrimidine and purine bases, preferably cytosine (C), thymine (T), and uracil (U), and adenine (A) and guanine (G), respectively. See Albert L. Lehninger, PRINCIPLES OF BIOCHEMISTRY, at 793-800 (Worth Pub. 1982). Indeed, the present invention contemplates any deoxyribonucleotide, ribonucleotide or peptide nucleic acid component, and any chemical variants thereof, such as methylated, hydroxymethylated or glucosylated forms of these bases, and the like. The polymers or oligomers may be heterogeneous or homogeneous in composition, and may be isolated from naturally occurring sources or may be artificially or synthetically produced. In addition, the nucleic acids may be deoxyribonucleic acid (DNA) or ribonucleic acid (RNA), or a mixture thereof, and may exist permanently or transitionally in single-stranded or double-stranded form, including homoduplex, heteroduplex, and hybrid states.

An “oligonucleotide” or “polynucleotide” is a nucleic acid ranging from at least 2, preferable at least 8, and more preferably at least 20 nucleotides in length or a compound that specifically hybridizes to a polynucleotide. Polynucleotides of the present invention include sequences of deoxyribonucleic acid (DNA) or ribonucleic acid (RNA), which may be isolated from natural sources, recombinantly produced or artificially synthesized and mimetics thereof. A further example of a polynucleotide of the present invention may be peptide nucleic acid (PNA) in which the constituent bases are joined by peptides bonds rather than phosphodiester linkage, as described in Nielsen et al., Science 254:1497-1500 (1991), Nielsen Curr. Opin. Biotechnol., 10:71-75 (1999). The invention also encompasses situations in which there is a nontraditional base pairing such as Hoogsteen base pairing which has been identified in certain tRNA molecules and postulated to exist in a triple helix. “Polynucleotide” and “oligonucleotide” are used interchangeably in this application.

An “array” is an intentionally created collection of molecules which can be prepared either synthetically or biosynthetically. The molecules in the array can be identical or different from each other. The array can assume a variety of formats, e.g., libraries of soluble molecules; libraries of compounds tethered to resin beads, silica chips, or other solid supports.

Nucleic acid library or array is an intentionally created collection of nucleic acids which can be prepared either synthetically or biosynthetically in a variety of different formats (e.g., libraries of soluble molecules; and libraries of oligonucleotides tethered to resin beads, silica chips, or other solid supports). Additionally, the term “array” is meant to include those libraries of nucleic acids which can be prepared by spotting nucleic acids of essentially any length (e.g., from 1 to about 1000 nucleotide monomers in length) onto a substrate. The term “nucleic acid” as used herein refers to a polymeric form of nucleotides of any length, either ribonucleotides, deoxyribonucleotides or peptide nucleic acids (PNAs), that comprise purine and pyrimidine bases, or other natural, chemically or biochemically modified, non-natural, or derivatized nucleotide bases. The backbone of the polynucleotide can comprise sugars and phosphate groups, as may typically be found in RNA or DNA, or modified or substituted sugar or phosphate groups. A polynucleotide may comprise modified nucleotides, such as methylated nucleotides and nucleotide analogs. The sequence of nucleotides may be interrupted by non-nucleotide components. Thus the terms nucleoside, nucleotide, deoxynucleoside and deoxynucleotide generally include analogs such as those described herein. These analogs are those molecules having some structural features in common with a naturally occurring nucleoside or nucleotide such that when incorporated into a nucleic acid or oligonucleotide sequence, they allow hybridization with a naturally occurring nucleic acid sequence in solution. Typically, these analogs are derived from naturally occurring nucleosides and nucleotides by replacing and/or modifying the base, the ribose or the phosphodiester moiety. The changes can be tailor made to stabilize or destabilize hybrid formation or enhance the specificity of hybridization with a complementary nucleic acid sequence as desired.

“Solid support”, “support”, and “substrate” are used interchangeably and refer to a material or group of materials having a rigid or semi-rigid surface or surfaces. In many embodiments, at least one surface of the solid support will be substantially flat, although in some embodiments it may be desirable to physically separate synthesis regions for different compounds with, for example, wells, raised regions, pins, etched trenches, or the like. According to other embodiments, the solid support(s) will take the form of beads, resins, gels, microspheres, or other geometric configurations.

Combinatorial Synthesis Strategy: A combinatorial synthesis strategy is an ordered strategy for parallel synthesis of diverse polymer sequences by sequential addition of reagents which may be represented by a reactant matrix and a switch matrix, the product of which is a product matrix. A reactant matrix is a 1 column by m row matrix of the building blocks to be added. The switch matrix is all or a subset of the binary numbers, preferably ordered, between 1 and m arranged in columns. A “binary strategy” is one in which at least two successive steps illuminate a portion, often half, of a region of interest on the substrate. In a binary synthesis strategy, all possible compounds which can be formed from an ordered set of reactants are formed. In most preferred embodiments, binary synthesis refers to a synthesis strategy which also factors a previous addition step. For example, a strategy in which a switch matrix for a masking strategy halves regions that were previously illuminated, illuminating about half of the previously illuminated region and protecting the remaining half (while also protecting about half of previously protected regions and illuminating about half of previously protected regions). It will be recognized that binary rounds may be interspersed with non-binary rounds and that only a portion of a substrate may be subjected to a binary scheme. A combinatorial “masking” strategy is a synthesis which uses light or other spatially selective deprotecting or activating agents to remove protecting groups from materials for addition of other materials such as amino acids.

Monomer: refers to any member of the set of molecules that can be joined together to form an oligomer or polymer. The set of monomers useful in the present invention includes, but is not restricted to, for the example of (poly)peptide synthesis, the set of L-amino acids, D-amino acids, or synthetic amino acids. As used herein, “monomer” refers to any member of a basis set for synthesis of an oligomer. For example, dimers of L-amino acids form a basis set of 400 “monomers” for synthesis of polypeptides. Different basis sets of monomers may be used at successive steps in the synthesis of a polymer. The term “monomer” also refers to a chemical subunit that can be combined with a different chemical subunit to form a compound larger than either subunit alone.

Biopolymer or biological polymer: is intended to mean repeating units of biological or chemical moieties. Representative biopolymers include, but are not limited to, nucleic acids, oligonucleotides, amino acids, proteins, peptides, hormones, oligosaccharides, lipids, glycolipids, lipopolysaccharides, phospholipids, synthetic analogues of the foregoing, including, but not limited to, inverted nucleotides, peptide nucleic acids, Meta-DNA, and combinations of the above. “Biopolymer synthesis” is intended to encompass the synthetic production, both organic and inorganic, of a biopolymer.

Related to a bioploymer is a “biomonomer” which is intended to mean a single unit of biopolymer, or a single unit which is not part of a biopolymer. Thus, for example, a nucleotide is a biomonomer within an oligonucleotide biopolymer, and an amino acid is a biomonomer within a protein or peptide biopolymer; avidin, biotin, antibodies, antibody fragments, etc., for example, are also biomonomers. Initiation Biomonomer: or “initiator biomonomer” is meant to indicate the first biomonomer which is covalently attached via reactive nucleophiles to the surface of the polymer, or the first biomonomer which is attached to a linker or spacer arm attached to the polymer, the linker or spacer arm being attached to the polymer via reactive nucleophiles.

Complementary or substantially complementary: Refers to the hybridization or base pairing between nucleotides or nucleic acids, such as, for instance, between the two strands of a double stranded DNA molecule or between an oligonucleotide primer and a primer binding site on a single stranded nucleic acid to be sequenced or amplified. Complementary nucleotides are, generally, A and T (or A and U), or C and G. Two single stranded RNA or DNA molecules are said to be substantially complementary when the nucleotides of one strand, optimally aligned and compared and with appropriate nucleotide insertions or deletions, pair with at least about 80% of the nucleotides of the other strand, usually at least about 90% to 95%, and more preferably from about 98 to 100%. Alternatively, substantial complementarity exists when an RNA or DNA strand will hybridize under selective hybridization conditions to its complement. Typically, selective hybridization will occur when there is at least about 65% complementary over a stretch of at least 14 to 25 nucleotides, preferably at least about 75%, more preferably at least about 90% complementary. See, M. Kanehisa Nucleic Acids Res. 12:203 (1984), incorporated herein by reference.

The term “hybridization” refers to the process in which two single-stranded polynucleotides bind non-covalently to form a stable double-stranded polynucleotide. The term “hybridization” may also refer to triple-stranded hybridization. The resulting (usually) double-stranded polynucleotide is a “hybrid.” The proportion of the population of polynucleotides that forms stable hybrids is referred to herein as the “degree of hybridization”.

Hybridization conditions will typically include salt concentrations of less than about 1M, more usually less than about 500 mM and less than about 200 mM. Hybridization temperatures can be as low as 5° C., but are typically greater than 22° C., more typically greater than about 30° C., and preferably in excess of about 37° C. Hybridizations are usually performed under stringent conditions, i.e. conditions under which a probe will hybridize to its target subsequence. Stringent conditions are sequence-dependent and are different in different circumstances. Longer fragments may require higher hybridization temperatures for specific hybridization. As other factors may affect the stringency of hybridization, including base composition and length of the complementary strands, presence of organic solvents and extent of base mismatching, the combination of parameters is more important than the absolute measure of any one alone. Generally, stringent conditions are selected to be about 5° C. lower than the thermal melting point ™ fro the specific sequence at s defined ionic strength and pH. The Tm is the temperature (under defined ionic strength, pH and nucleic acid composition) at which 50% of the probes complementary to the target sequence hybridize to the target sequence at equilibrium.

Typically, stringent conditions include salt concentration of at least 0.01 M to no more than 1 M Na ion concentration (or other salts) at a pH 7.0 to 8.3 and a temperature of at least 25° C. For example, conditions of 5×SSPE (750 mM NaCl, 50 mM NaPhosphate, 5 mM EDTA, pH 7.4) and a temperature of 25-30° C. are suitable for allele-specific probe hybridizations. For stringent conditions, see for example, Sambrook, Fritsche and Maniatis. “Molecular Cloning A laboratory Manual” 2nd Ed. Cold Spring Harbor Press (1989) and Anderson “Nucleic Acid Hybridization” 1st Ed., BIOS Scientific Publishers Limited (1999), which are hereby incorporated by reference in its entirety for all purposes above.

Hybridization probes are nucleic acids (such as oligonucleotides) capable of binding in a base-specific manner to a complementary strand of nucleic acid. Such probes include peptide nucleic acids, as described in Nielsen et al., Science 254:1497-1500 (1991), Nielsen Curr. Opin. Biotechnol., 10:71-75 (1999) and other nucleic acid analogs and nucleic acid mimetics. See U.S. Pat. No. 6,156,501 filed Apr. 3, 1996.

Hybridizing specifically to: refers to the binding, duplexing, or hybridizing of a molecule substantially to or only to a particular nucleotide sequence or sequences under stringent conditions when that sequence is present in a complex mixture (e.g., total cellular) DNA or RNA.

Probe: A probe is a molecule that can be recognized by a particular target. In some embodiments, a probe can be surface immobilized. Examples of probes that can be investigated by this invention include, but are not restricted to, agonists and antagonists for cell membrane receptors, toxins and venoms, viral epitopes, hormones (e.g., opioid peptides, steroids, etc.), hormone receptors, peptides, enzymes, enzyme substrates, cofactors, drugs, lectins, sugars, oligonucleotides, nucleic acids, oligosaccharides, proteins, and monoclonal antibodies.

Target: A molecule that has an affinity for a given probe. Targets may be naturally-occurring or man-made molecules. Also, they can be employed in their unaltered state or as aggregates with other species. Targets may be attached, covalently or noncovalently, to a binding member, either directly or via a specific binding substance. Examples of targets which can be employed by this invention include, but are not restricted to, antibodies, cell membrane receptors, monoclonal antibodies and antisera reactive with specific antigenic determinants (such as on viruses, cells or other materials), drugs, oligonucleotides, nucleic acids, peptides, cofactors, lectins, sugars, polysaccharides, cells, cellular membranes, and organelles. Targets are sometimes referred to in the art as anti-probes. As the term targets is used herein, no difference in meaning is intended. A “Probe Target Pair” is formed when two macromolecules have combined through molecular recognition to form a complex.

Effective amount refers to an amount sufficient to induce a desired result. mRNA or mRNA transcripts: as used herein, include, but not limited to pre-mRNA transcript(s), transcript processing intermediates, mature mRNA(s) ready for translation and transcripts of the gene or genes, or nucleic acids derived from the mRNA transcript(s). Transcript processing may include splicing, editing and degradation. As used herein, a nucleic acid derived from an mRNA transcript refers to a nucleic acid for whose synthesis the mRNA transcript or a subsequence thereof has ultimately served as a template. Thus, a cDNA reverse transcribed from an mRNA, a cRNA transcribed from that cDNA, a DNA amplified from the cDNA, an RNA transcribed from the amplified DNA, etc., are all derived from the mRNA transcript and detection of such derived products is indicative of the presence and/or abundance of the original transcript in a sample. Thus, mRNA derived samples include, but are not limited to, mRNA transcripts of the gene or genes, cDNA reverse transcribed from the mRNA, cRNA transcribed from the cDNA, DNA amplified from the genes, RNA transcribed from amplified DNA, and the like.

A fragment, segment, or DNA segment refers to a portion of a larger DNA polynucleotide or DNA. A polynucleotide, for example, can be broken up, or fragmented into, a plurality of segments. Various methods of fragmenting nucleic acid are well known in the art. These methods may be, for example, either chemical or physical in nature. Chemical fragmentation may include partial degradation with a DNase; partial depurination with acid; the use of restriction enzymes; intron-encoded endonucleases; DNA-based cleavage methods, such as triplex and hybrid formation methods, that rely on the specific hybridization of a nucleic acid segment to localize a cleavage agent to a specific location in the nucleic acid molecule; or other enzymes or compounds which cleave DNA at known or unknown locations. Physical fragmentation methods may involve subjecting the DNA to a high shear rate. High shear rates may be produced, for example, by moving DNA through a chamber or channel with pits or spikes, or forcing the DNA sample through a restricted size flow passage, e.g., an aperture having a cross sectional dimension in the micron or submicron scale. Other physical methods include sonication and nebulization. Combinations of physical and chemical fragmentation methods may likewise be employed such as fragmentation by heat and ion-mediated hydrolysis. See for example, Sambrook et al., “Molecular Cloning: A Laboratory Manual,” 3rd Ed. Cold Spring Harbor Laboratory Press, Cold Spring Harbor, N.Y. (2001) (“Sambrook et al.) which is incorporated herein by reference for all purposes. These methods can be optimized to digest a nucleic acid into fragments of a selected size range. Useful size ranges may be from 100, 200, 400, 700 or 1000 to 500, 800, 1500, 2000, 4000 or 10,000 base pairs. However, larger size ranges such as 4000, 10,000 or 20,000 to 10,000, 20,000 or 500,000 base pairs may also be useful.

Polymorphism refers to the occurrence of two or more genetically determined alternative sequences or alleles in a population. A polymorphic marker or site is the locus at which divergence occurs. Preferred markers have at least two alleles, each occurring at frequency of greater than 1%, and more preferably greater than 10% or 20% of a selected population. A polymorphism may comprise one or more base changes, an insertion, a repeat, or a deletion. A polymorphic locus may be as small as one base pair. Polymorphic markers include restriction fragment length polymorphisms, variable number of tandem repeats (VNTR's), hypervariable regions, minisatellites, dinucleotide repeats, trinucleotide repeats, tetranucleotide repeats, simple sequence repeats, and insertion elements such as Alu. The first identified allelic form is arbitrarily designated as the reference form and other allelic forms are designated as alternative or variant alleles. The allelic form occurring most frequently in a selected population is sometimes referred to as the wildtype form. Diploid organisms may be homozygous or heterozygous for allelic forms. A diallelic polymorphism has two forms. A triallelic polymorphism has three forms. Single nucleotide polymorphisms (SNPs) are included in polymorphisms.

Single nucleotide polymorphism (SNPs) are positions at which two alternative bases occur at appreciable frequency (>1%) in the human population, and are the most common type of human genetic variation. The site is usually preceded by and followed by highly conserved sequences of the allele (e.g., sequences that vary in less than 1/100 or 1/1000 members of the populations). A single nucleotide polymorphism usually arises due to substitution of one nucleotide for another at the polymorphic site. A transition is the replacement of one purine by another purine or one pyrimidine by another pyrimidine. A transversion is the replacement of a purine by a pyrimidine or vice versa. Single nucleotide polymorphisms can also arise from a deletion of a nucleotide or an insertion of a nucleotide relative to a reference allele.

Genotyping refers to the determination of the genetic information an individual carries at one or more positions in the genome. For example, genotyping may comprise the determination of which allele or alleles an individual carries for a single SNP or the determination of which allele or alleles an individual carries for a plurality of SNPs. A genotype may be the identity of the alleles present in an individual at one or more polymorphic sites.

III. Large Scale Genotyping Methods

Natural selection is at the heart of adaptive evolution. Characteristics of human populations are shaped by their responses to pathogens, diet, climate and other selective pressures. Identifying regions of the genome under directional selection has important implications for understanding our evolution as a species. Natural selection is one of the factors that influence linkage disequilibrium (LD), the non-random association of alleles at adjacent loci in the genome. Flanking regions may be swept into fixation along with the selected variants, resulting in LD over long distances (Kaplan, N L, Hudson, R R, Langley, C H (1989) The “hitchhiking effect” revisited. Genetics. 123:887-899). To date, identification of variants postulated to be under positive natural selection has been limited to those occurring in a handful of special genes, tested in multiple populations to uncover geographic structure. This is akin to the “candidate gene” approach in the search for disease genes (F. S. Collins, M. S. Guyer, A. Chakravarti, Variations on a theme: cataloguing human DNA sequence variation. Science 278, 1580 (1997)). The most famous example is the natural selection of blood groups conferring resistance to malaria (Cavalli-Sforza, L. L, Menozzi, P., and Piazza, A. (1994) The history and geography of human genes. Princeton University Press, Princeton, N.J. 1994). Variants in other human genes have subsequently been proposed to have been targets of selective pressure (Osier M V, Pakstis A J, Soodyall H, Comas D, Goldman D, Odunsi A, Okonofua F, Parnas J, Schulz L O, Bertranpetit J, Bonne-Tamir B, Lu R B, Kidd J R, Kidd K K. A Global Perspective on Genetic Variation at the ADH Genes Reveals Unusual Patterns of Linkage Disequilibrium and Diversity. Am J Hum Genet 2002 Jun. 5;71(1) [epub ahead of print]; Harris, E. E. and Hey, J. X chromosome evidence for ancient human histories. Proc. Natl. Acad. Sci. USA. 96:3320-3324 (1999); Gelermter, J., Cubells, J F, Kidd, J R, Pakstis, A J, Kidd K K (1999) Population studies of polymorphisms of the serotonin transporter protein gene Am. J. Hum. Genet. 88: 61-66; Gilad, Y., Rosenberg, S., Przeworski, M., Lancet, D. and Skorecki, K. Evidence for positive selection and population structure at the human MAO-A gene. Proc. Natl. Acad. Sci. USA. 99:862-867 (2002); Rana B K, Hewett-Emmett D, Jin L, Chang B H, Sambuughin N, Lin M, Watkins S, Bamshad M, Jorde L B, Ramsay M, Jenkins T, Li W H. High polymorphism at the human melanocortin 1 receptor locus. Genetics 1999 Apr;151(4):1547-57; Hedrick, P. W. and Kim, T. J. Genetics of Complex Polymorphisms: parasites and maintenance of the Major Histocompatibility Complex variation. In Evolutionary Genetics, Cambridge University Press, Cambridge, UK pp204-234 (2000); Hamblin, M. T., Thompson, E. E. and Di Rienzo, A. Complex signatures of natural selection at the Duffy Blood group locus. Am. J. Hum. Genet. 70:369-383 (2002); Hamblin, M. T. and Di Rienzo, A. Detection of the signature of natural selection in humans: evidence from the Duffy blood group locus. Am. J. Hum. Genet. 66:1669-1679 (2000); Fullerton, S. M., Bartoszewicz, A., Ybazeta, G., Horikawa, Y., Bell, G. I., Kidd, K. K., Cox, N. J., Hudson, R. R. and Di Rienzo, A. Geographic and haplotype structure of candidate type 2 diabetes-susceptibility variants at the calpain-10 locus. Am. J. Hum. Genet. 70:1096-1106 (2002)), but the numbers of loci examined have been relatively few. How many other regions of the human genome contain variants whose frequencies have been radically altered as a result of selective pressure?

A genome-wide search for such variants requires large-scale analysis of variation across the genomes of multiple individuals from different populations, i.e. the creation of a geographic map with many thousands of markers. The simplest variations to study are single-nucleotide polymorphisms (SNPs), because they are abundant and less prone to mutation than microsatellites. Public efforts have identified over 2 million common human SNPs, however the scoring of these SNPs is labor-intensive, requiring a substantial amount of automation. Creation of a highly parallel genotyping platform could facilitate progress in such large-scale studies.

In one aspect of the invention, methods are provided to detect genes or genomic regions of biological importance. In some embodiments, genomic samples from different (at least two) populations (a population, as used herein, refers to a race, people from a geographic region, etc.) are examined for differences in allel frequencies. In preferred embodiments, differences in SNP allele frequencies are examined. A whole genome assay (WGA) developed at Affymetrix, Inc. (Santa Clara, Calif.), described in, e.g., U.S. Provisional Applicaiton, attorney docket number 3504, and U.S. Pat. No. 09/916,135, provide an efficient methods for genotyping a large number of SNPs in a complex DNA sample. Other methods for genotyping may also be suitable for the methods of the invention.

The differences in frequencies may be evaluated using the Fst statistic (see below). The degree of geographic structure can be estimated by the F_{ST} statistic (Weir, B. S. Genetic Data Analysis II, Sinauer Associates, Inc Sunderland, Mass. (1996)), which varies from 0 to 1.

Regions of the genome that are under selective pressure may be identified according to the allele difference, e.g., calculating F_{ST} values for all SNPs and looking for SNPs with exceptionally high values, such as greater than 0.4, 0.5, 0.6, 0.7, or 0.8.

The genomic region under selective pressure may be identified as having potential biological functions, such as disease resistance.

In some embodiments, genes residing in the genomic regions may be identified by, for example, examining gene annotation databases.

The genomic region or the genes residing in the genomic regions may be further examined and used as therapeutical targets, as they have already been shown by human history to be targets of regulation.

One of skill in the art would appreciate that the methods of the invention is not limited to any particularly species. The methods can be used detect genomic regions of interest in many organisms such as human, other animals, plants, etc.

IV. Example

The following example shows exemplary embodiments of various aspects of the invention.

Introduction

Genetic studies aimed toward understanding the molecular basis of complex human phenotypes are the subject of considerable discussion. One hypothesis proposes that the key to mapping causal variants is to exploit regions of linkage disequilibrium (LD) across the genome by genotyping a very dense set of markers. The simplest markers to study are single-nucleotide polymorphisms (SNPs), because they are abundant and less prone to mutation than microsatellites. The extent of LD is known to vary widely across the genome and between populations(Reich, D. E., Cargill, M., Bolk, S., Ireland, J., Sabeti, P. C., Richter, D. J., Lavery, T., Kouyoumjian, R., Farhadian, S. F., Ward, R. & Lander, E. S. Linkage disequilibrium in the human genome. Nature 411, 199-204 (2001); Gabriel, S. B., Schaffner, S. F., Nguyen, H., Moore, J. M., Roy, J., Blumenstiel, B., Higgins, J., DeFelice, M., Lochner, A., Faggart, M., Liu-Cordero, S. N., Rotimi, C., Adeyemo, A., Cooper, R., Ward, R., Lander, E. S., Daly, M. J. & Altshuler, D. The structure of haplotype blocks in the human genome. Science 296, 2225-2229 (2002)), leading to differing estimates of the number of genome-wide SNPs necessary to capture a significant portion of this LD. Others are skeptical that LD mapping, even with large numbers of SNPs, will work for more than a handful of special cases. The controversy is not limited to SNP numbers, but also to the choice of SNPs and their role in representing human diversity. Proponents of the common disease-common haplotype hypothesis suggest that genotyping haplotype-defining SNPs will decrease the numbers of SNPs needed for complex genetic studies, however, the numbers of SNPs required may still be high. Public efforts have identified over 2 million common human SNPs, however the scoring of these SNPs is labor-intensive, requiring a substantial amount of automation. Creation of a highly parallel genotyping platform could therefore facilitate progress in such large-scale studies.

There are two significant bottlenecks to achieving high parallelism: the requirement for locus-specific amplification of each SNP, and the requirement for locus-specific allele discrimination. These bottlenecks were overcame by devising a generic sample preparation method that uses a small number of oligonucleotide primers, coupled to allele discrimination on synthetic DNA microarrays. Arrays containing >500,000 probe sequences have been constructed; these arrays access large quantities of genetic information in a highly parallel fashion by relying on the specific hybridization of nucleic acids in samples to complementary sequences on the array. While it has been shown that arrays of increasingly higher and higher information content can be synthesized, a principle challenge is how to present genomic DNA to the array and ultimately derive accurate allelic information about the sample. As the number of unique genomic base pairs (i.e. complexity) in the target increases, opportunities for cross-hybridization and non-specific signals increase, while accuracy decreases. It is therefore necessary to present subsets, or fractions, of the genome to the array in order to derive meaningful and specific signal.

In this example, high-density synthetic DNA arrays were used to sequence individual nucleotides in approximately 1×10⁷ bp (10 Mb). All of the above studies made significant advances in the preparation of reduced representations of the genome, yet they could score only a portion of the genomic variations residing in those fractions.

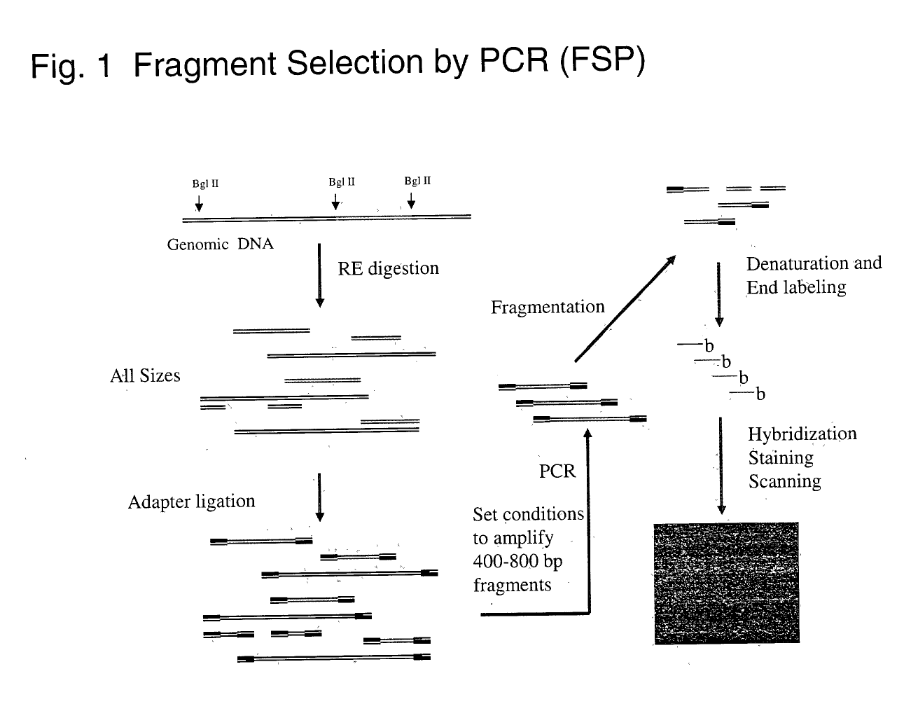

The overall strategy begins with in silico prediction of SNPs residing in desired genomic fractions and synthesis of these SNP-containing fragments onto high-density microarrays. Following biochemical fractionation that mirrors the in silico fractionation, target is hybridized to arrays and SNPs are genotyped by allele-specific hybridization. The biochemical fractionation method we devised, called “Fragment Selection by PCR” or FSP, is shown in FIG. 1. The total genomic DNA was digested with one of several restriction enzymes and ligated the digested DNA with adaptors recognizing the cohesive four bp overhangs. All fragments resulting from restriction enzyme digestion, regardless of size, are substrates for adaptor ligation. A generic primer, which recognizes the adaptor sequence, is used to amplify ligated DNA fragments. PCR reaction conditions were optimized to selectively and reproducibly amplify fragments in the 400-800 bp size range, the same size range used by TSC, thereby achieving both fractionation of the genome and maximization of TSC SNP content.

Results

Target Hybridizations

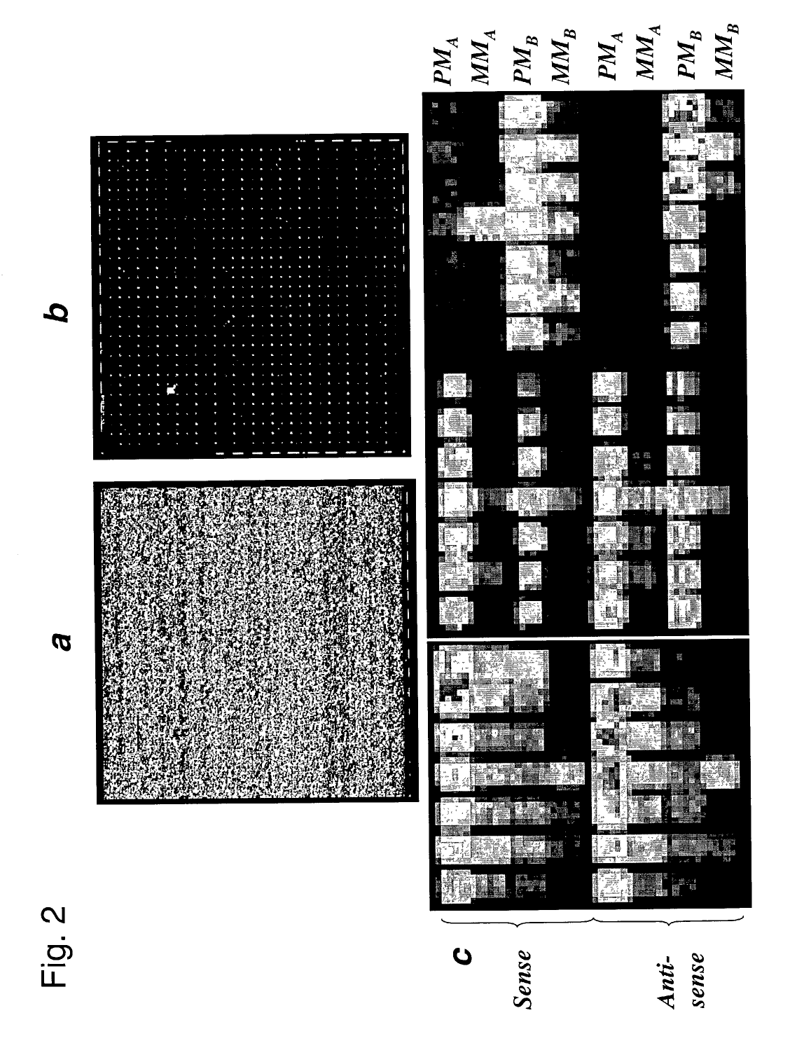

Targets generated by FSP were labeled and hybridized to the arrays. Each fraction represents approximately 4×10⁷ bp of genomic DNA. An image of a representative array hybridized with one fraction shows robust signal intensities (FIG. 2a). In contrast, hybridization of total human genomic DNA (3.2×10⁹ bp) results in low signals (FIG. 2b), a substantial portion of which is noise. A close-up view of a SNP “block” hybridized with DNA from three different individuals representing all three genotypes is shown in FIG. 2c. Hybridization signals which allow interpretation of genotypes are clearly visible by eye, demonstrating the feasibility of our generic approach.

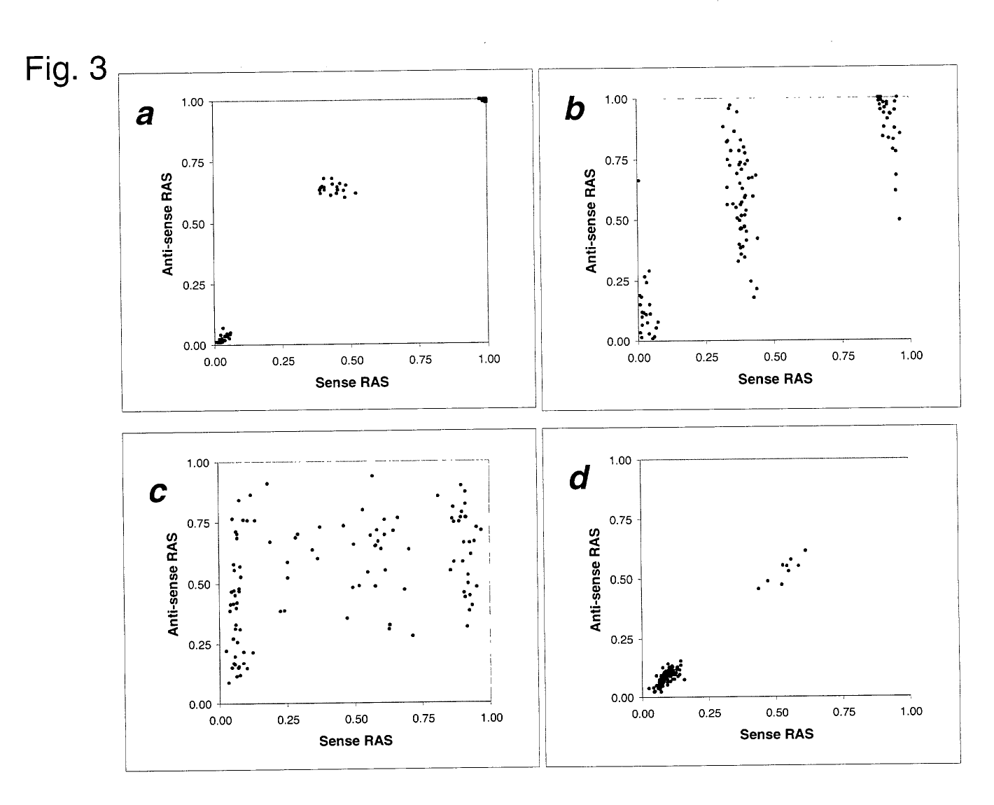

Algorithm Training an automated scoring process was developed for calling genotypes. The training data was derived from 108 ethnically diverse DNA samples. The relative allele signal (RAS) values for each SNP was calculated on both sense and antisense strands and plotted them for all 108 individuals in two dimensions. Some SNPs show three clearly defined clusters (FIG. 3a), while others show more diffuse clusters (FIG. 3b), or no clear clusters at all (FIG. 3c). For those SNPs having lower minor allele frequencies, the genotypes fall into only two clusters, with the minor allele homozygote cluster being absent (FIG. 3d). Following graphic visualization of clusters derived from RAS values in two dimensions, an algorithm was developed to classify these points into two or three clusters and evaluate the quality of classification with the average silhouette width, s. As s approaches 1.0, clusters are tight and well-separated, while low values of s, e.g. <0.5, are derived from poorly clustering SNPs.

A series of heuristics were developed for ranking the SNPs according to their clustering properties. Of the 71,931 SNPs assessed in this experiment, ˜20% or 14,548 met the most stringent criteria. Only SNPs that formed three clusters were scored; therefore many SNPs that formed only two good clusters did not meet the cut-off criteria.

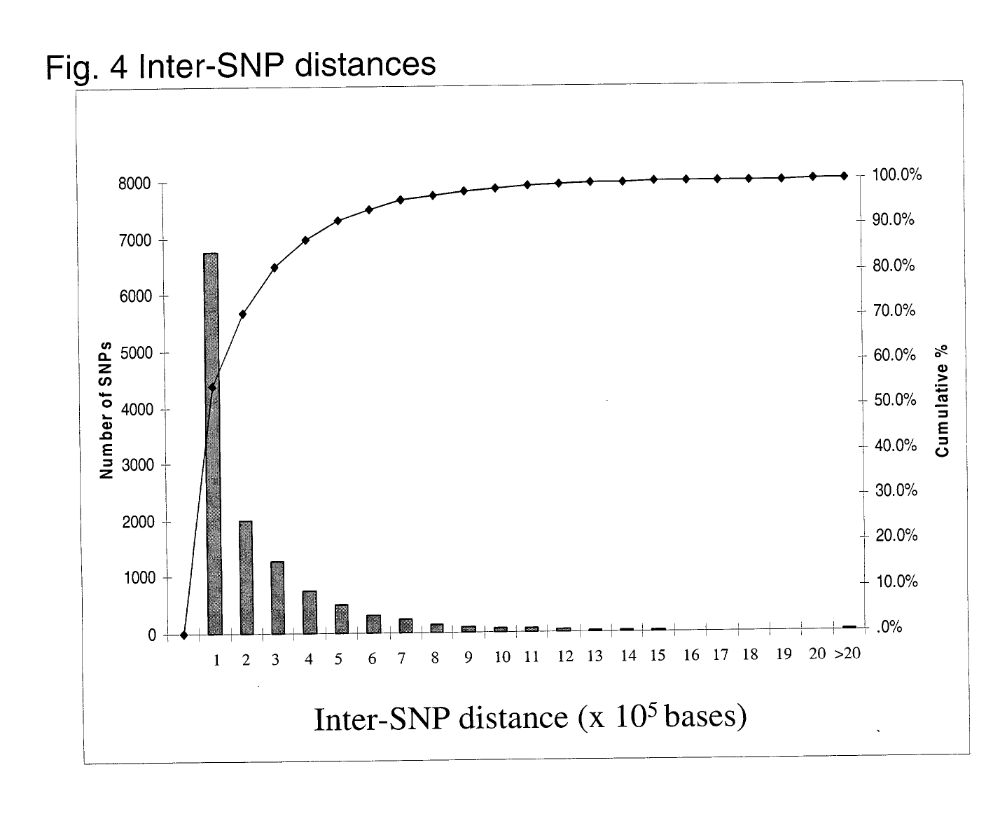

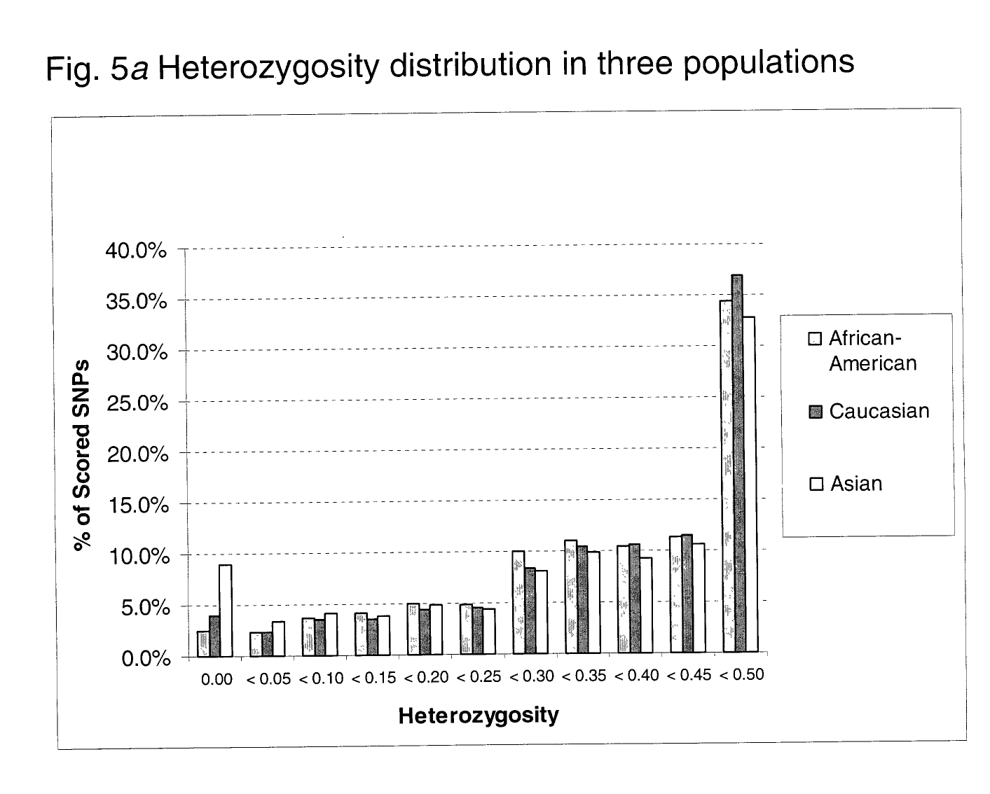

The mean and median heterozygosity of the 14,548 markers across 108 individuals is 0.386 and 0.421, respectively (theoretical maximum=0.50), indicating that these markers should be highly informative in a variety of ethnic populations studied here. The SNPs were mapped on the human genome sequence by TSC. The distribution of inter-SNP distances between markers is shown in FIG. 4; the mean and median intermarker distances are 174 kb and 80.8 kb, respectively. Of these markers, 5058 are spaced at distances of 50 kb or less; 3868 are spaced at distances of 25 kb or less. This density allows mapping in familial linkage studies and is predicted to capture some proportion of linkage disequilibrium in the genome.

Reproducibility and Accuracy

Genetic studies typically involve genotyping hundreds of samples, thus all genotyping methods must interrogate SNPs reproducibly across DNA samples. The average genotype call rate is 95.1%±1.2%, demonstrating a high level of reproducibility. The accuracy of our genotype calls were determined in two ways: through the use of genotypes obtained by independent genotyping methods, and by dideoxynucleotide sequencing of discordant genotype calls. The accuracy of the genotypes was determined to be >99.5%.

Allele Frequency Determination

The allele frequencies of 13,647 SNPs in DNA from 60 unrelated individuals comprising three human populations: African-American, Caucasian and Asian, were determined. A comparison of the allele frequencies derived from a set of 20 Caucasians versus a set of 38 Caucasians shows a high correlation (R²=0.96), indicating that sampling of 20 individuals provides reasonably stable estimates of allele frequencies for these SNPs in that population. Furthermore, allele frequencies for 313 of our SNPs were also determined by TSC as part of the allele frequency project (AFP) and these allele frequencies agree well with ours.

Of the 13,647 SNPs interrogated, the vast majority were polymorphic in all three populations. This is consistent with expectations, as the training set consisted of an ethnically diverse panel of individuals. In this analysis, there were 343, 535 and 1219 markers in the African-American, Caucasian and Asian samples, respectively, which were monomorphic (i.e. zero heterozygosity). Of these, 100 were monomorphic in both African-Americans and Asians (but not Caucasians), 81 were monomorphic in African-Americans and Caucasians (but not in Asians) and 236 were monomorphic in both Asians and Caucasians (but not African-Americans).

Population Studies

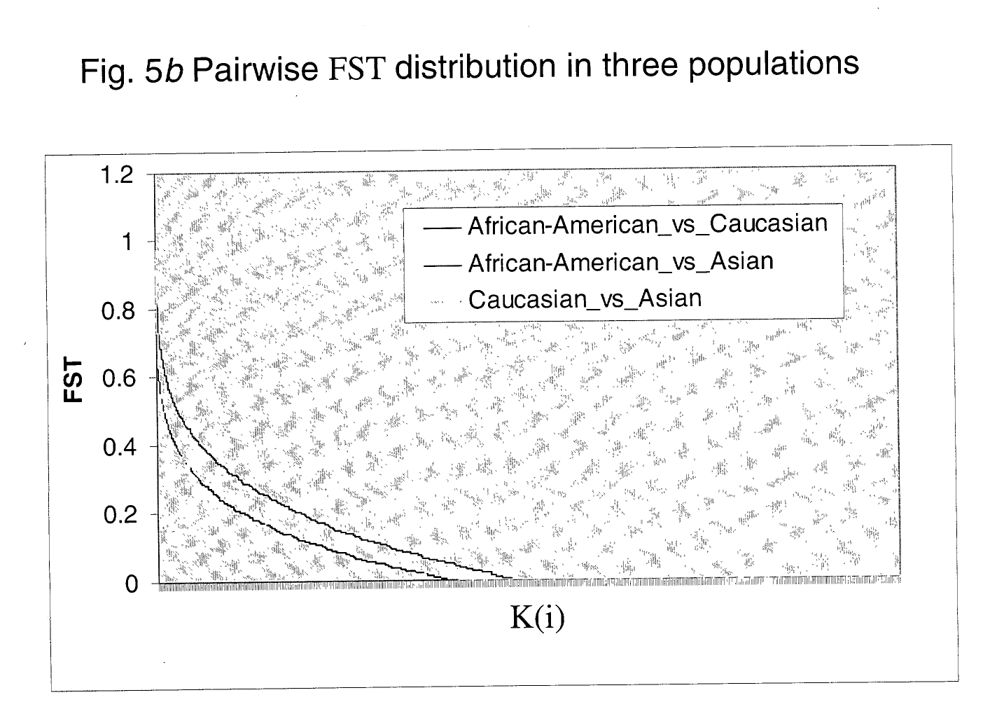

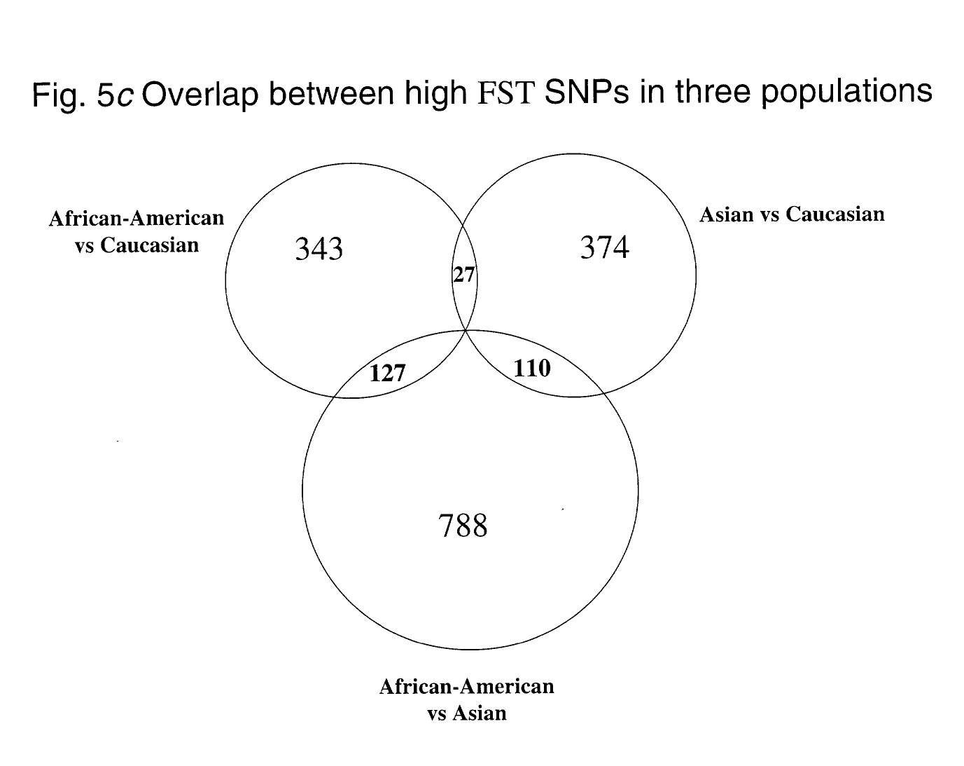

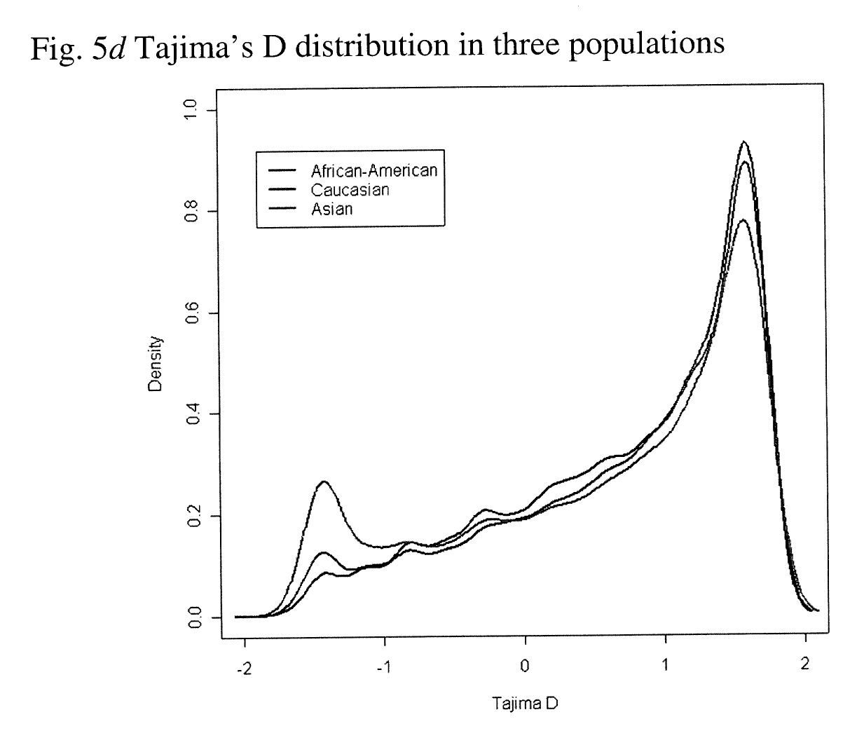

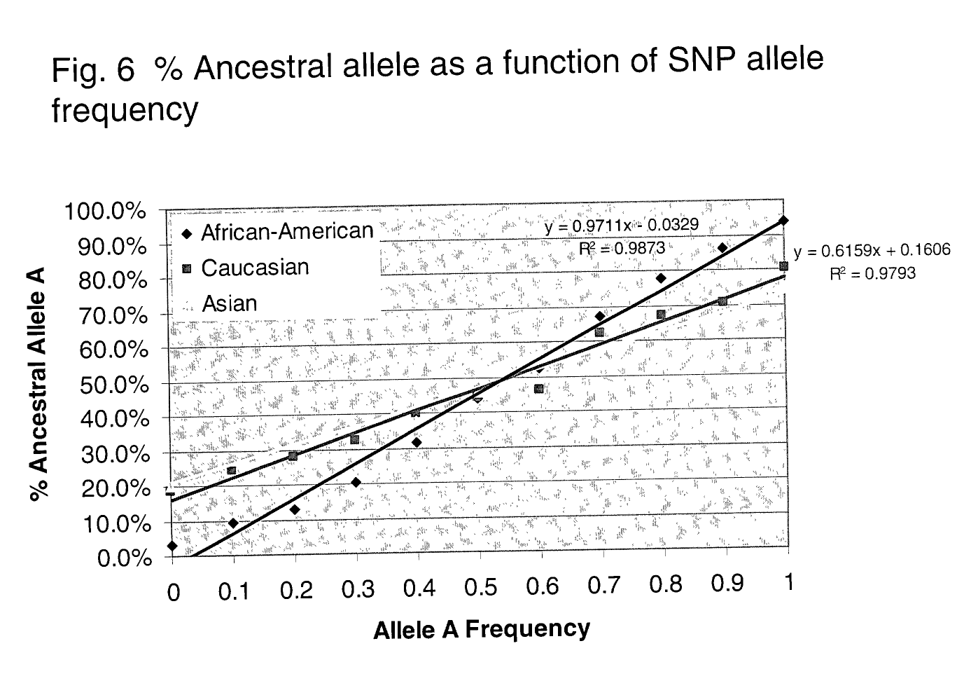

The allele frequency spectrum in a given population bears the signatures of its history. Forces such as natural selection, random genetic drift, demographic events such as population bottlenecks or expansions, or some combination of all of these, manifest their effects on populations. Demographic events and genetic drift are expected to have genome-wide effects on the allele frequency spectrum, while natural selection exerts its effects on a few specific loci in the genome. In an effort to begin to understand the similarities and differences amongst the African-American, Caucasian and Asian populations, genome-wide parameters for the three populations were determined (FIGS. 5a-c). Following this analysis, a subset of SNPs showing extreme differences in allele frequency amongst the three populations were closely examined, and these findings were correlated with ancestral allele information and estimates of departures from neutrality (see below, Table 1, FIG. 5d; FIGS. 6-8).

Genome-wide Parameters

The distribution of marker heterozygosity in the three populations is shown in FIG. 5a. The mean heterozygosity of the markers is 0.348, 0.354 and 0.322 in the African-American, Caucasian and Asian samples, respectively, indicating that the vast majority of SNPs will be informative in the populations studied here. the F_{ST} statistic was calculated (Weir, B. S. Genetic Data Analysis II, Sinauer Associates, Inc Sunderland, Mass. (1996).), which is an estimate of the geographic structure between two populations, for each SNP. F_{ST} values vary from 0 to 1; as allele frequency differences between populations become more pronounced, F_{ST} values increase. Distributions of F_{ST} for each pairwise population are shown in FIG. 5b for 13,647 SNPs. The mean F_{ST} values are 0.061, 0.094 and 0.065 for the African-American versus Caucasian, African-American versus Asian and Caucasian versus Asian comparisons, respectively, indicating that the majority of markers show very small inter-population frequency differences, consistent with the ethnic diversity of the training set from which these SNPs were ascertained. These mean values are consistent with F_{ST} distributions previously reported for a smaller number of loci¹⁷. The comparison between African-American and Asian allele frequencies was shifted significantly toward higher F_{ST} values relative to the other two comparisons (FIG. 5a). The observed genome-wide shift in distribution beween the Asian and African-American samples could have been caused by random genetic drift and/or demographic events such as population bottlenecks or expansion.

Uncovering Potential Sites of Natural Selection

In contrast to demographic events which are likely to manifest their effects on a genome-wide level, directional selection is confined to specific loci. The results from this example show that while most SNPs demonstrate small or moderate allele frequency differences among the three populations, there are a small subset of SNPs whose allele frequencies differ significantly in one population versus the other two. Previous simulations predict that, if random genetic drift is the only force responsible for allele frequency differences between populations, only 1% of observed F_{ST} values will be ≧0.4 (Bowcock, A. M., Kidd, J. R., Mountain, J. L., Hebert, J. M., Carotenuto, L., Kidd, K. K. & Cavalli-Sforza, L. L. Drift, admixture, and selection in human evolution: a study with DNA polymorphisms. Proc. Nat. Acad. Sci. 88, 839-843 (1991)). In this example, 2.6% of the SNPs have an F_{ST} value of ≧0.4, nearly three times the expected number. These SNPs may provide valuable information regarding human population history. High F_{ST} values could occur by chance (ie random genetic drift, bottlenecks or expansions) or because a variant confers a selective advantage in that population (ie selective sweeps or balancing selection).

In many cases, there is a biological rationale for testing whether a variant has a high F_{ST} value, however, even in the absence of a priori biological evidence, one might be able to uncover putative sites that have undergone natural selection by looking at the most extreme tail of the F_{ST} distribution. Identification of these putative sites could serve as starting points for formulating biological hypotheses, and hopefully lead to experimental testing and ultimately, to proof or refutation of the hypotheses. In this example, SNPs with high F_{ST} values in a comparison of all three populations were closely examined. 108 SNPs with F_{ST} values >0.5 and 354 SNPs with F_{ST} values >0.4 were found; the highest value is 0.893 (Table 1). A subset of these, 28 SNPs with F_{ST} values >0.6 (Table 1), were examined. It is possible that the high F_{ST} values we observed could have been due to stochastic error, given the relatively small number of individuals (N=20) sampled from these populations. However, SNPs with truly high F_{ST} values should be flanked by neighboring SNPs which also show high F_{ST} values. In many cases our assay captured a SNP <10 kb away from the initial high F_{ST} SNP, and most of these SNPs also had significantly high F_{ST} values (Table 1), indicating that they were not likely due to sampling errors. Furthermore, the “direction” of allelic differences among the three populations is also consistent among the closely linked SNPs, suggesting that the locus travels as a “block”, ie the nearby SNPs are likely to be in linkage disequilibrium. For example, four SNPs located in close proximity to each other on chromosome 4q34.3 in the VEGF-C gene show F_{ST} values of 0.286-0.610. In contrast, the next closest SNPs on either side of the block show F_{ST} values of zero (248.7 kb away) and 0.133 (291 kb away), consistent with the decay of F_{ST} values as a function of distance. In several cases, TSC allele frequency information was available for a nearby SNP not captured by our assay; the data sets agree in all but two cases (unpublished data). Public databases and genome annotations were used to identify transcripts in the vicinity of the SNPs with high F_{ST} values. While the exact map locations of the SNPs, along with their annotations, three of the high F_{ST} SNPs were located in well-annotated genes. These genes are involved in important biological pathways such as lymphangiogenesis (VEGF-C), signal transduction (dok5) and DNA repair (XRCC4). There are also SNPs that fall near or within transcripts of unknown function, or in regions of the genome that currently lack annotation.

While it is possible that high F_{ST} values could result from SNPs that have undergone natural selection, an alternate explanation is that allele frequencies have been skewed as a result of a population bottleneck. There is evidence for a single, major “out of africa” bottleneck shared by Caucasians and Asians. If all the SNPs with high F_{ST} values were caused by a common bottleneck following the migration from Africa, then one would expect those SNPs to have high F_{ST} values in both non-African populations studied here. The overlap between high F_{ST} SNPs for three pairwise comparisons was examined and it was found that this was not the case (FIG. 5c). While most of the high F_{ST} SNPs are derived from differences between the African and non-African populations, there are a small number that derive from differences between the two non-African populations. This observation, coupled with the highly significant shift in F_{ST} distribution FIG. 5b) in the African-American versus Asian comparison, is consistent with the hypothesis that the Asian population experienced demographic or selective event(s) distinct from the other two populations.

TABLE 1
Characteristics of SNPs with F_{ST} values > 0.6. Allele frequencies in three populations, chromosomal positions,
annotations and gene information were obtained from the TSC, UCSC and ENSEMBL genome browsers as of June 27, 2002.

| | | | | | | Distance | | Name | |
|---|---|---|---|---|---|---|---|---|---|
| | | | | | | of | FST | RefSeq or | |
| | | | | | | closest | of | Genbank | |
| SNP | | pAfrican | | | Chromosome | SNP | closest | Transcripts | Distance |
| ID | FST | American | pCaucasian | pAsian | position | (kb) | SNP | in region | (kb) |
| 12407 | 0.893 | 1.00 | 1.00 | 0.10 | NA | 535.15 | 0.760 | NAG-13 | 0.00 |
| 843191 | 0.772 | 0.27 | 0.97 | 0.00 | X | | | | |
| 609713 | 0841 | 0.15 | 1.00 | 1.00 | X | 304.52 | 0.342 | L1 Retrotransposon | 0.00 |
| 883586 | 0.759 | 1.00 | 0.98 | 0.20 | 2p24.1 | 0.10 | 0.382 | Visinin-like peptide | 366.70 |
| 240790 | 0.454 | 0.20 | 0.25 | 0.88 | | | | | |
| 66220 | 0.748 | 0.88 | 0.11 | 0.00 | 14q24.3 | 202.09 | 0.052 | Melanoma cDNA | 1.21 |
| 274926 | 0.702 | 0.88 | 0.17 | 0.00 | 20q13.2 | 91.78 | 0.041 | DOK5 | 0.00 |
| 56501 | 0.689 | 0.87 | 0.94 | 0.09 | 15q26.1 | 0.31 | 0.282 | Synaptic vesicle | 312.73 |
| | | | | | | | | protein 2B | |
| 70430 | 0.688 | 0.08 | 0.73 | 1.00 | X | 118.11 | 0.042 | Chordin-like mRNA | |
| 898013 | 0.686 | 1.00 | 0.56 | 0.03 | X | 144.79 | 0.079 | Melanoma antigen | 54.26 |
| 61517 | 0.680 | 0.31 | 1.00 | 1.00 | X | 136.58 | 0.099 | PHEX | 0.00 |
| 67262 | 0.679 | 0.15 | 0.93 | 0.94 | X | 69.16 | 0.433 | FMR2 | 0.00 |
| 525494 | 0.433 | 0.44 | 1.00 | 0.93 | | | | | |
| 519349 | 0.676 | 0.75 | 0.05 | 0.00 | 18q22.2 | 268.79 | 0.158 | Testis EST | 3.56 |
| 954810 | 0.675 | 0.73 | 0.03 | 0.00 | X | 0.04 | 0.158 | | |
| 54826 | 0.664 | 0.05 | 0.15 | 0.88 | 16q22.3 | 6.42 | 0.158 | Heparanase | 87.56 |
| 472245 | 0.653 | 0.95 | 0.85 | 0.13 | | 6.48 | 0.653 | | |
| 578939 | 0.651 | 0.30 | 0.98 | 1.00 | 7q32.1 | 12.95 | 0.110 | Neuroblastoma EST | 37.10 |
| 573362 | 0.644 | 0.98 | 0.95 | 0.25 | 5p15.33 | 0.10 | 0.199 | Ileal mucosa cDNA | 0.00 |
| 9279 | 0.632 | 0.35 | 1.00 | 1.00 | NA | 201.00 | 0.162 | NA | NA |
| 39181 | 0.626 | 0.15 | 0.74 | 1.00 | NA | 0.32 | 0.066 | NA | NA |
| 608238 | 0.626 | 0.04 | 0.83 | 0.88 | X | 79.52 | 0.000 | ribosomal protein | 23.76 |
| | | | | | | | | L28 | |
| 41257 | 0.624 | 1.00 | 0.87 | 0.21 | X | 115.33 | 0.426 | Guanylate cyclase | 186.65 |
| | | | | | | | | 2F | |
| 619537 | 0.426 | 0.43 | 0.15 | 0.88 | | | | | |
| 58237 | 0.620 | 0.80 | 0.10 | 0.05 | 8p12 | 248.45 | 0.193 | Synovial fibroblast | 0.00 |
| | | | | | | | | functional retro- | |
| | | | | | | | | transposon | |
| 49480 | 0.610 | 0.30 | 0.97 | 0.98 | NA | 0.02 | 0.286 | NA | NA |
| 49481 | 0.286 | 0.55 | 0.93 | 0.98 | | 0.02 | 0.610 | | |
| 355816 | 0.469 | 0.53 | 0.03 | 0.00 | | 55.92 | 0.534 | | |
| 714589 | 0.534 | 0.28 | 0.90 | 0.95 | 4q34.3 | 55.92 | 0.469 | VEGFC | 0.00 |
| 56677 | 0.608 | 0.12 | 0.83 | 0.90 | X | 13.53 | 0.208 | FMR2 | 0.00 |
| 47637 | 0.603 | 0.65 | 0.83 | 0.00 | X | 122.91 | 0.327 | kidney EST | 194.60 |
| 65066 | 0.602 | 0.08 | 0.40 | 0.95 | 5q15 | 22.41 | 0.052 | alpha- | 14.81 |
| | | | | | | | | mannosidase | |
| 367998 | 0.600 | 0.38 | 1.00 | 1.00 | X | 99.83 | 0.571 | Androgen receptor | 42.90 |
| 843745 | 0.571 | 0.22 | 0.80 | 1.00 | | | | | |

TABLE 2
Summary results for genotypes in chimp and gorilla. FSP assay was
performed on chimpanzee and gorilla genomic DNA, along with
human DNA as a control, and genotypes called on 14,558 SNPs.
Absolute numbers of genotype calls and their percentages are shown.

| | Human | Chimp | Gorilla |
|---|---|---|---|
| Number SNPs called A | 4401 | 5475 | 5061 |
| Number SNPs called B | 4431 | 5495 | 5156 |
| Number SNPs called AB | 4731 | 256 | 238 |
| Number No Calls | 995 | 3332 | 4103 |
| Total Calls | 13563 | 11226 | 10455 |
| Number Attempted Calls | 14558 | 14558 | 14558 |
| CallRate | 93.20% | 77.10% | 71.80% |
| % A | 32.40% | 48.80% | 48.40% |
| % B | 32.67% | 48.95% | 49.32% |
| % AB | 34.88% | 22.80% | 2.27% |

Table 3 X Chromosome vs Autosomal SNP Comparison for F_{ST} and Tajima Parameters

TABLE 3
X chromosome vs Autosomal SNP comparison for F_{ST} and
Tajima parameters

| | | Mean | | | |
|---|---|---|---|---|---|
| | Mean X | Autosome | d.f. | t-value | p-value |
| F_{ST} | | | | | |
| 2wayF_{ST} | 0.095 | 0.06 | 321.912 | 3.811 | 0.00016662 |
| (Af vs Ca) | | | | | |
| 2wayF_{ST} | 0.124 | 0.093 | 322.929 | 2.745 | 0.006392 |
| (Af vs As) | | | | | |
| 2wayF_{ST} | 0.067 | 0.064 | 324.507 | 0.358 | 0.7209 |
| (Ca vs As) | | | | | |
| 3wayF_{ST} | 0.131 | 0.091 | 321.631 | 3.923 | 0.0001 |
| Tajima's D | | | | | |
| D (African- | 0.379 | 0.748 | 325.639 | −6.2934 | 1.00E−09 |
| American) | | | | | |
| D (Caucasian) | 0.098 | 0.774 | 323.519 | −10.0791 | <2.2e−16 |
| D (Asian) | 0.093 | 0.585 | 325.074 | −6.981 | 1.66E−11 |

Ancestral Allele Determination

SNPs are “mutations” that have arisen once during evolution; to determine which of the two alleles represents the ancestral state, we determined genotypes on chimpanzee and gorilla genomic DNA samples. Chimpanzee and gorilla DNA differs from human by 1.5% and 2.1%, respectively. The result from the example indicates that chimpanzee and gorilla genotypes can be called on 77.1% and 71.8% of the human SNPs, respectively (Table 2). The overwhelming majority of markers are homozygous in both great ape species (Table 2), consistent with the recent evolutionary history of SNPs. There are a small number of heterozygous SNPs that may represent shared (and thus very ancient) polymorphisms, however data from a larger number of great apes is necessary to assess Hardy-Weinberg equilibrium of these markers. We assigned ancestral alleles only to SNPs that met the following criteria: SNPs that were homozygous in both chimpanzee and gorilla, and that gave the same genotype call in both species. A total of 8386 SNPs were assigned. Consistent with theoretical predictions, previous results for 214 SNPs in an ethnically diverse set of samples showed that the most frequent allele is not always ancestral(Hacia, J. G., Fan, J. B., Ryder, O., Jin, L., Edgemon, K., Ghandour, G., Mayer, R. A., Sun, B., Hsie, L., Robbins, C. M., Brody, L. C., Wang, D., Lander, E. S., Lipshutz, R., Fodor, S. P. & Collins, F. S. Determination of ancestral alleles for human single-nucleotide polymorphisms using high-density oligonucleotide arrays. Nat Genet. 22, 164-7 (1999)). These results in the present study were extended by examining this relationship for a larger number of SNPs in three human populations. The distribution of the chimpanzee and gorilla (ie ancestral) alleles was plotted as a function of SNP allele frequency in the African-American, Caucasian and Asian populations and found in each case a strong positive correlation; the higher the SNP allele frequency, the higher the proportion of the ancestral allele (FIG. 6). The slopes of the Caucasian and Asian populations are 0.62 and 0.52, respectively. These data indicate that in these two populations the ancestral allele is not always the most frequent allele; ie about 20% of the time, the newer allele has become more frequent in these populations, consistent with previous studies^{33,32}. In contrast, the slope of the curve in African-Americans is 0.97, indicating a nearly one-to-one correlation between ancestral state and allele frequency. In this population, regardless of relative allele frequency, the most frequent allele is almost always the ancestral allele, contrary to theoretical predictions.

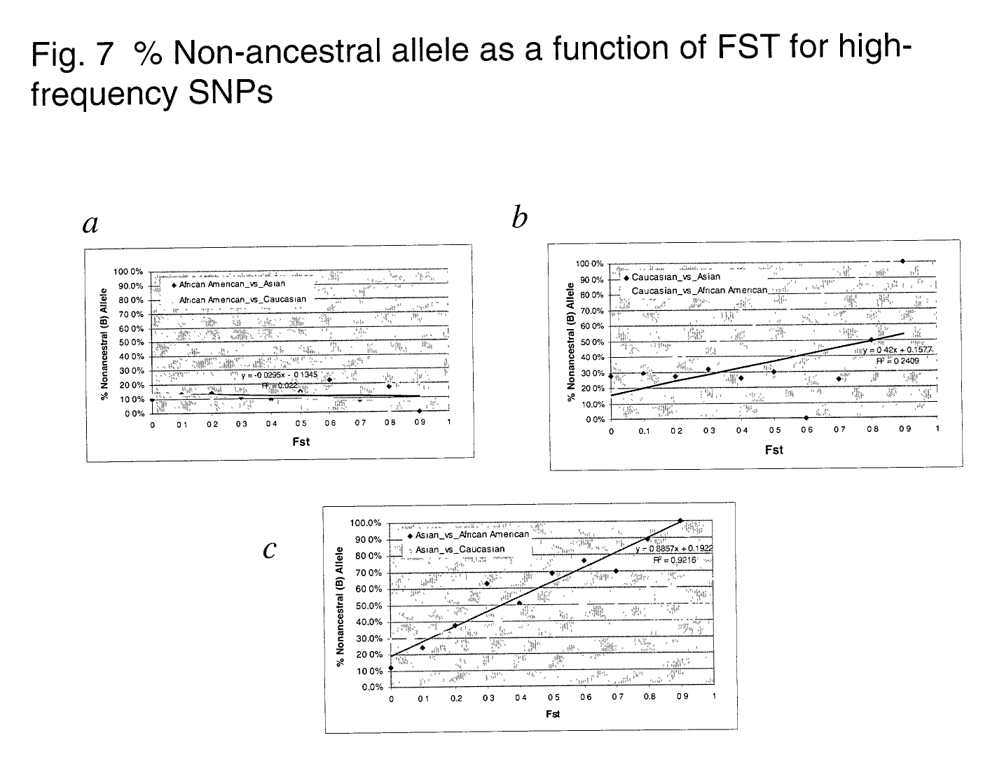

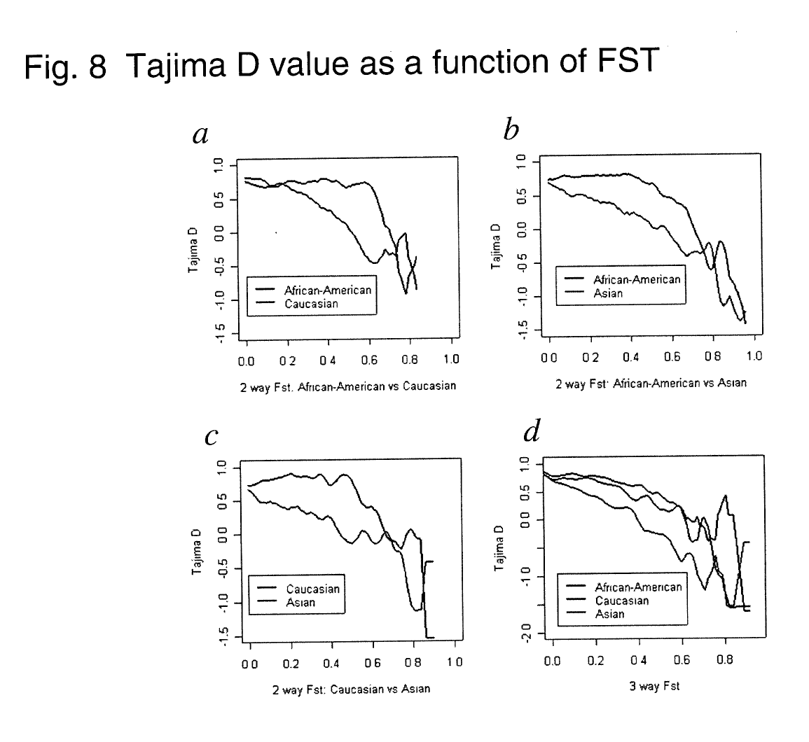

The new “mutations” i.e. non-ancestral alleles, could have reached high frequencies (ie near-fixation) in the non-African populations by random genetic drift, population bottlenecks, expansions or natural selection. Whether this set of high-frequency, non-ancestral alleles were more (or less) likely to have high F_{ST} values was determined, i.e. to show geographic structure. Each population was examined for SNPs with allele frequencies >0.8, determined the F_{ST} values for each pairwise population comparison, then determined the percentage of SNPs corresponding to the non-ancestral allele. A striking positive correlation between the non-ancestral state and F_{ST} values in the Caucasian and Asian high-frequency SNPs (FIGS. 7b-c) was found. In contrast, high frequency SNPs in African-Americans showed no such correlation with F_{ST} values as a function of non-ancestral state (FIG. 7a).

Departure from the Neutral Theory: Tajima's D Statistic

The neutral theory of evolution maintains that the majority of mutations (e.g. SNPs) are either strongly deleterious, or of no selective importance(Kimura, M. Evolutionary rate at the molecular level. Nature217, 624-626 (1968)). Deleterious mutations are rapidly eliminated from the population, thus most of the variation between populations are the result of neutral mutations, random genetic drift and recombination. In this model, natural selection plays no role in shaping diversity. Tests such as Tajima's can be used to test for departures from neutrality and uncover sites that may have been subject to demographic forces and/or natural selection (Tajima, F. Statistical method for testing the neutral mutation hypothesis by DNA polymorphism. Genetics123, 585-595 (1989).). The Tajima's D statistic is negative when there is an excess of recent, rare mutations, i.e. in situations where a locus has undergone natural selection (e.g. a selective sweep) or population growth. Tajima's D statistic is positive when there is an excess of mutants at intermediate frequency, i.e. when a locus has been involved in balancing selection, or a population bottleneck. We calculated the Tajima D statistic for 13,647 SNPs in each of the populations and plotted it as a density distribution (FIG. 5d). Mean Tajima D values are 0.75, 0.77 and 0.58 for the African-American, Caucasian and Asian populations, respectively. The genome-wide distribution clearly shows departures from the neutral theory for all three populations (FIG. 5d). The Tajima's D values for the high F_{ST} SNPs were specifically examined in Table 1 and a high preponderance of negative values was noted, suggesting that these SNPs may have been influenced by natural selection, population growth, or a combination of both. To determine whether there was any relationship between values of Tajima's D and F_{ST}, we plotted the Tajima's D value for each F_{ST} bin in three pairwise comparisons (FIGS. 8a-c) as well as a three-way comparison (FIG. 8d). In all cases, there is a clear correlation with the Tajima D statistic; as F_{ST} values increase, Tajima's D becomes negative. Furthermore, the shape of the curves differs between populations, indicating variation in the relative contributions of demographic and/or selective forces to the observed allele frequency differences. Although the majority of SNPs (i.e. those with low F_{ST} values) are associated with positive Tajima's D values, revealing the effects of a population bottleneck or balancing selection, the small percentage of SNPs with high F_{ST} values probably arose as a result of population growth or natural selection, rather than a population bottleneck.

Comparison of X Chromosome SNPs with Autosomal SNPs

It is noted that many high F_{ST} SNPs are located on the X chromosome (Table 1). To determine whether SNPs on the X chromosome showed properties significantly different from autosomal SNPs, we compared the F_{ST} and Tajima D values for all 316 X chromosome SNPs with those from a random set of 316 autosomal SNPs (Table 3). Two-way F_{ST} values reveal significant differences between X and autosomal SNPs for both African-American vs Caucasian and African-American vs Asian comparisons (p <0.00016 and p <0.0064, respectively), but not in the Caucasian vs Asian comparison. The 3-way F_{ST} differences are also significant (p <0.0001). Differences in Tajima's D values between X chromosome and autosomal SNPs in each of the three populations were also highly significant (Table 3). While the mean Tajima D value of the X chromosome SNPs is significantly higher in African-Americans compared to non-African-Americans, the Asian population has a significantly lower mean Tajima D value in autosomal SNPs relative to the Caucasian and African-American population. The observation that SNPs on the X chromosome have significantly higher F_{ST} values than the autosomal SNPs reveals a type of “locus-specific effect” that is consistent with a selective, rather than demographic (ie genome-wide), event.

Discussion

The example shows the simultaneous genotyping of 14,548 SNPs without the use of locus-specific PCR. This approach can be extended to genotype even larger number of SNPS, e.g. >100,000. In silico SNP prediction and size fractionation of the genome one aspect of the approach; it can be extended to include additional restriction enzyme fractions, regardless of whether size selection is accomplished through FSP or by other means. With the WGA approach, one can use increasing numbers of enzyme fractions to genotype large numbers of SNPs and approach ultra-high genome mapping densities. The generic approach requires 1 restriction-enzyme-specific oligonucleotide for each genomic subfraction, plus one generic oligonucleotide that amplifies all SNPs. The interrogation of 71,931 SNPs in the present study required only four primers. Furthermore, a single microarray can genotype simultaneously ˜10,000 SNPs by reducing the number of probes per SNP; such reduction can be achieved without loss of accuracy (unpublished data). This approach not only scales to larger numbers of SNPs, but scales to other complex organisms as well. As draft genome sequence is completed for other genomes such as mouse, we envision a SNP discovery effort mirroring that of TSC, namely the use of restriction enzyme and size fractionation. Implementation of these protocols for discovery of SNPs in complex organisms will enable immediate use of WGA technology and thus facilitate acceleration of genetic studies in model organisms.

This genotyping technology was used to rapidly obtain genome-wide allele frequency data on a variety of populations. This large data set, in conjunction with allele frequency data from TSC and other public databases, takes an important step in creating a large-scale catalogue of human diversity. In addition to revealing genome-wide similarities and differences, allele frequency data can uncover interesting departures from neutral models of evolution and can help us begin to unravel the complex forces that have shaped the history of human populations.

The tools can now be applied across a variety of other scientific disciplines to address many pressing genetic questions, especially those requiring a dense set of variants spaced across the genome. For example, with this technology, it is feasible to rapidly determine allele frequencies in other geographic populations, to create high-resolution haplotype maps, and to map regions of LD across the genome, all at unprecedented resolution. With these tools in hand, it should soon be possible to embark upon paradigm genetic studies aimed at uncovering the molecular basis of complex human phenotypes and to better understand the evolutionary history.

Methods

Array Design. In order to genotype as many TSC SNPs as possible on the fewest numbers of arrays, we designed the arrays to interrogate only those SNPs predicted to be amplified by our biochemical assays. Completion of the draft human genome sequence made it possible to conduct in silico digests of total genomic DNA, identify the desired size fragments, and predict which SNPs should be present on those fragments. We excluded fragments containing repetitive sequences within the tiled region; these represented about 25-30% of TSC SNPs. A series of 11 arrays were synthesized containing sequence from 71,931 unique SNPs present in three different genomic subfractions (EcoRI, BglII and XbaI). A total of 56 probes were synthesized for each SNP (FIG. 2c). For each SNP, probes (25-mers) were synthesized, spanning seven positions along both strands of the SNP-containing sequence, with the SNP position in the center, (position zero) as well as at −4, −2, −1, +1, +3, +4. Probes were synthesized for both sense and antisense strands. Four probes were synthesized for each of the 7 positions: a perfect match (PM) for each of the two SNP alleles (A, B) and a one-base central mismatch (MM) for each of the two alleles (FIG. 2c). Normalized discrimination, calculated as (PM−MM)/(PM+MM) is a measure of sequence specificity, and is used in the detection filter of the genotype calling algorithm³⁶.

DNA Samples. Samples used in the training set included 24 individuals from the polymorphism discovery panel (PD1-24), along with 6 unrelated CEPH individuals, 20 African-Americans, 20 Asians and 20 Caucasians from the TSC Allele Frequency panels, all available through the Coriell Institute for Medical Research as part of the National Institute of General Medical Sciences Human Genetic Mutant Cell Repository at http://umdnj.edu/locus/nigms/. Chimp and gorilla samples were obtained from Coriell.

Target Preparation. Total genomic DNA (250 ng) is incubated with 20 units of EcoRI, BglII or XbaI restriction endonuclease (New England Biolabs) at 37° C. for 4 hrs. Following heat inactivation at 75° C. for 20 min, the digested DNA is incubated with 0.25 uM adaptors and DNA ligase (NEB) in standard ligation buffer (NEB) at 16° C. for 4 hrs. The sample is incubated at 95° C. for 5 min to inactivate the enzyme. Target amplification is performed with ligated DNA and 0.5 μM primer in PCR Buffer II (Perkin Elmer) with 2.5 mM MgCl₂, 250 μM dNTPs and 50 units of Taq polymerase (Perkin Elmer). Cycling is conducted as follows: 95° C./10 min followed by 20 cycles of 95° C./10s, 58° C./15 sec, 72° C./15sec, followed by 25 cycles of 95° C./20 sec, 55° C./15 sec, 72° C./15. Final extension is performed at 72° C. for 7 minutes. The amplification products are concentrated with a YM30 column (Microcon) centrifuged at 14,000 rfc for 6 min. Column is washed twice with 400 μl H₂O, respun at 14,000 rfc, inverted and the sample recovered in a clean tube by centrifuging at 3000 rfc for 3 min. The sample is digested with 0.045 units DNase (Affymetrix) and 0.5 units calf intestinal phosphatase (Gibco) in RE Buffer #4 (NEB) at 37° C. for 30 minutes. Enzymes are inactivated at 95° C. for 15 min. Samples are labeled with 15-20 units Terminal deoxytransferase (Promega), 18 μM biotinylated ddATP (NEN) in TdT buffer (Promega) at 37° C. for 4 hrs. Following heat inactivation at 95° C. for 10 min, samples are injected into microarray cartridges and hybridized overnight following manufacturer's directions (Affymetrix). Microarrays are washed in a fluidics station (Affymetrix) using 0.6×SSPET, followed by a three-step staining protocol. First the arrays are incubated with 10 μg/ml streptavidin (Pierce), followed by a wash with 6×SSPET, followed by 10 μg/ml biotinylated anti-streptavidin (Vector Lab), 10 ug/ml streptavidin-phycoerythrin conjugate (Molecular Probes) and a final wash of 6×SSPET. Microarrays are scanned according to manufacturer's directions (Affymetrix). It is estimated that each of the enzyme fractions used in this study to have a complexity of ˜42 Mb. This estimation is affected by several factors: accuracy of genome sequence used for in silico fractionations, efficiency of adaptor ligation and amplification; the theoretical value for complexity based on the draft human genome sequence (April 2001 release) was calculated and uniform amplification of target fragments was assumed.

Genomic DNA was digested with NsiI, and subjected to electrophoresis on 0.6% agarose gels. Bands were excised in the desired size range, DNA extracted with QiaQuick gel extraction kit, and quantitated. Fragments were then subjected to ligation and amplification as described above.

Algorithm Training. Samples used in the training set included 24 individuals from the polymorphism discovery panel (PD1-24), along with 6 unrelated CEPH individuals, 20 African-Americans, 20 Asians and 20 Caucasians from the TSC Allele Frequency panels, all available through the Coriell Institute for Medical Research. RAS is calculated as the median of the ratios Ai/(Ai+Bi), where Ai and Bi are signals of A and B alleles of the ith probe quartet. The silhouette width is a relative measure of the difference between the distance of a data point to the nearest neighbor group and the distance of the data point to other data points in the same group. Silhouette widths range from −1 to 1; the larger the silhouette width, the better the classification from a clustering point of view. Briefly, the algorithm includes a signal detection filter based on Wilcoxon's signed rank test, classification using a modification of partitioning around medoids (PAM) and the computation of several quality scores. SNPs were selected based on the following criteria: those that formed three clusters with s>0.7, showed separation of RAS medians between clusters >0.2, and 90% of the samples passed the detection filter. Following clustering, boundaries around the clusters were detemined for the purposes of assigning incoming points to one of the clusters, i.e., making genotype calls. The method used in this report assigns a center point for each cluster. The coordinates of the center are the sense and antisense medians of all points in a cluster. The genotype call boundary is determined by the Euclidian distance to the center, and the call zone is then restricted to 80% of that distance.

Accuracy Determination. Reproducibility was determined on a set of 38 Caucasian samples, genotyped as incoming data on clusters defined by the training set. The percentage of successful genotype calls (call rate) was averaged over 38 samples and ranged from 91.5-97.3%. Reference genotypes were obtained for approximately 900 SNPs assayed using single-base extension (SBE) technology and compared these genotypes to those generated by WGA. We found a concordance rate of 99.1% in these markers over 38 samples (total of 33,111 calls compared). Ten SNPs accounted for >50% of the 311 discordant genotypes. de novo nucleotide sequence for these 10 SNPs across individuals exhibiting discordant genotypes were obtained, and it was found that WGA genotype calls were concordant with sequence data 44% of the time. Thus, the accuracy of WGA genotype calls is most likely >99.5%. Genotypes for 65 SNPs across 7 individuals were compared with data derived from high-resolution scanning of chromosome 2. Of 287 calls compared between the two datasets, there was only one discordant genotype (i.e. concordance rate=99.6%). Additional confidence in the accuracy of our genotype calls was obtained indirectly by examining genomic DNA isolated from two complete hydatidiform moles (CHM). These products of abnormal conception arise from the fertilization of an empty ovum by a single sperm, resulting in complete duplication of the haploid paternal genome. Genotypes are expected to be homozygous for all markers. Both tumors showed 0.4% heterozygosity, consistent with expectations of a completely duplicated haploid genome, while a control sample of normal placenta showed 35.3% heterozygosity (unpublished data). Of the 205 SNPs synthesized on two or more arrays and captured by different enzyme fractions, the concordance rate for genotype calls was 99.5% across 30 individuals. Lastly, the Mendelian inheritance (MI) error rate was determined by genotyping samples from 40 CEPH families for a subset of the markers (7005 SNPs) and determined the average MI error rate to be <1.0% (unpublished data).

Allele Frequency Studies.

Samples from the three populations (denoted TSC DNA panels) are available from Coriell. A total of 313 SNPs overlapped our data set and that of TSC allele frequency project (AFP). A scatterplot of the allele frequencies in the two data sets showed a correlation coefficient R^{2=0.90}. Only 13,647 out of the 14,548 were interrogated because we omitted SNPs captured by more than one enzyme fraction, and those SNPs whose genotypes were called in fewer than 15 out of the 20 individuals in any one of the three ethnic groups.

URL's. The SNP Consortium Website is snp.cshl.org

Allele frequency panel samples (snp.cshl.org/allele_frequency_project/panels.html). Coriell Institute for Medical Research as part of the National Institute of General Medical Sciences Human Genetic Mutant Cell Repository at umdnj.edu/locus/nigms/. Genome annotations were those obtained from the TSC (www.cshl.org), from UCSC Genome Browser (www.) and from ENSEMBL (www.ensembl.org) as of Jun. 27, 2002.

It is to be understood that the above description is intended to be illustrative and not restrictive. Many variations of the invention will be apparent to those of skill in the art upon reviewing the above description. The scope of the invention should be determined with reference to the appended claims, along with the full scope of equivalents to which such claims are entitled. All cited references, including patent and non-patent literature, are incorporated herewith by reference in their entireties for all purposes.

What is claimed is:

1. A method for identifying a genomic region under natural selection comprising;
genotyping at least 5,000 SNPs in at least two populations;
determining difference of allele frequencies between the populations to identify at least one SNP with a Fst value of at least 0.3
identifying the genomic region where the at least one SNP resides as a putative genomic region under natural selection.

2. A method for identifying a genomic region of biological significance comprising;
genotyping at least 5,000 SNPs in at least two populations;
determining difference of allele frequencies between the populations to identify at least one SNP with a Fst value of at least 0.3;
identifying the genomic region where the at least one SNP resides as a putative genomic region of biological significance.

3. A method for identifying a genomic region as potential pharmaceutical target comprising;
genotyping at least 5,000 SNPs in at least two populations;
determining difference of allele frequencies between the populations to identify at least one SNP with a Fst value of at least 0.3;
identifying the genomic region where the at least one SNP resides as a putative genomic region of pharmaceutical target.

4. A method for identifying a gene under natural selection comprising;
genotyping at least 5,000 SNPs in at least two populations;
determining difference of allele frequencies between the populations to identify at least one SNP with a Fst value of at least 0.3
identifying the genomic region where the at least one SNP resides; and
identifying a gene in the genomic region as under natural selection.

5. A method for identifying a gene as pharmaceutical target comprising;
genotyping at least 5,000 SNPs in at least two populations;
determining difference of allele frequencies between the populations to identify at least one SNP with a Fst value of at least 0.3;
identifying the genomic region where the at least one SNP resides; and
identifying a gene in the genomic region as a pharmaceutical target.
